(19) United States
(12) Patent Application Publication
(10) Pub. No.: US 2026/0069321 A1
(43) Pub. Date: Mar. 12, 2026

(54) MODULAR TULIP FOR USE WITH ORTHOPEDIC SCREW

(71) Applicant: MiRus LLC, Marietta, GA (US)

(72) Inventor: Nikolay Laubert, Marietta, GA (US)

(21) Appl. No.: 19/316,379

(22) Filed: Sep. 2, 2025

Related U.S. Application Data
(60) Provisional application No. 63/692,819, filed on Sep. 10, 2024.

(51) Int. Cl. A61B 17/70

(52) U.S. Cl. CPC A61B 17/7037

(57) ABSTRACT

A polyaxial pedicle screw arrangement that can be temporarily locked in place a polyaxial pedicle screw arrangement in position during a surgical procedure.

### REFERENCED APPLICATIONS

The present application claims priority on U.S. patent application Ser. No. 63/692,819 filed Sep. 10, 2024, which is fully incorporated herein by reference.

### FIELD OF DISCLOSURE

The disclosure relates generally to medical devices and medical device applications, more particularly to orthopedic devices, and still more particularly to a polyaxial pedicle screw arrangement for use in spinal implant applications.

### BACKGROUND

When implanting screws and rods in the spine, there exists a need to temporarily lock in place a polyaxial pedicle screw arrangement during a surgical procedure. Polyaxial pedicle screw arrangements are known in the art as illustrated in U.S. Pat. Nos. 8,876,869; 9,186,187; 9,980,753; 11,344,336; 11,638,597; US 2017/0172627; US 2012/0085373; and EP 2221013, all of which are fully incorporated herein by reference.

In view of the current state of the art of spinal implants, there is a need for a polyaxial pedicle screw arrangement that can be temporarily locked in place during a surgical procedure.

### SUMMARY OF THE DISCLOSURE

The present disclosure is direct to a medical device in the form of a polyaxial pedicle screw arrangement for use in spinal implant applications. The present invention is directed to polyaxial pedicle screw arrangement that can be temporarily locked in place during a surgical procedure. The temporary locking feature of the polyaxial pedicle screw arrangement enables a user to perform one or more repositioning maneuvers of the polyaxial pedicle screw arrangement during a surgical procedure (e.g., derotation, compression, distraction etc.).

In accordance with a non-limiting aspect of the present disclosure, there is provided a polyaxial pedicle screw arrangement for use in spinal implant applications. The polyaxial pedicle screw arrangement includes a provisional/temporary fastening arrangement that enables the polyaxial pedicle screw arrangement to be locked and unlocked during a surgical procedure. The provisional/temporary fastening arrangement of the polyaxial pedicle screw arrangement is configured to be activated and deactivated by the use of one or more medical instruments that are configured to engage the polyaxial pedicle screw arrangement in a particular region of the polyaxial pedicle screw arrangement to cause the provisional/temporary fastening arrangement to lock the polyaxial pedicle screw in a current orientation. In one non-limiting embodiment, the one or more medical instruments are configured to engage the polyaxial pedicle screw arrangement in a particular region of the polyaxial pedicle screw arrangement and optionally apply pressure to the provisional/temporary fastening arrangement and/or to cause movement of one or more components of the provisional/temporary fastening arrangement to move to the locked position and cause the polyaxial pedicle screw arrangement to be locked in its current orientation. The polyaxial pedicle screw arrangement can optionally be configured such that when the one or more medical instruments are partially or fully disengaged from the provisional/temporary fastening arrangement and/or cause movement of one or more components of the provisional/temporary fastening arrangement, the provisional/temporary fastening arrangement is caused to move to an unlocked position which thereby enables the polyaxial pedicle screw arrangement to move in various axial orientations. In one non-limiting arrangement, the one or more medical instruments are configured to cause a pressure and/or a compressive force on one or more portions of the provisional/temporary fastening arrangement to cause movement of one or more components provisional/temporary fastening arrangement to thereby convert (e.g., temporarily convert or permanently convert) the polyaxial pedicle screw arrangement into a monoaxial-behaving screw. In another non-limiting arrangement, the one or more medical instruments are configured to cause a pressure and/or a compressive force on two or more regions or portions of the provisional/temporary fastening arrangement (e.g., 2-8 regions or portions of the provisional/temporary fastening arrangement and all values and ranges therebetween) to cause movement of one or more components provisional/temporary fastening arrangement to thereby convert (e.g., temporarily convert or permanently convert) the polyaxial pedicle screw arrangement in to a monoaxial-behaving screw.

In accordance with another and/or alternative non-limiting aspect of the present disclosure, there is provided a polyaxial pedicle screw arrangement wherein the polyaxial pedicle screw arrangement includes a tulip arrangement, an upper pressure cap, a lower pressure cap/flexible collet, and a pedicle screw. In one non-limiting embodiment, the one or more medical instruments are configured to cause a pressure and/or a compressive force on one or more regions or portions of the upper pressure cap (e.g., 1-8 regions or portions of the upper pressure cap, 2 regions of the upper pressure cap, 4 regions of the upper pressure cap, etc.) to cause movement of one or more regions of the upper pressure cap, a lower pressure cap/flexible collet to thereby convert (e.g., temporarily convert or permanently convert) the polyaxial pedicle screw arrangement in to a monoaxial-behaving screw. In another and/or alternative non-limiting embodiment, the one or more medical instruments are configured to cause a pressure and/or a compressive force on one or more corner regions of the upper pressure cap (e.g., 2-8 corner regions of the upper pressure cap, 2 corner regions of the upper pressure cap, 4 corner regions of the upper pressure cap, etc.) to cause movement of one or more corner regions of the upper pressure cap 60 to thereby convert (e.g., temporarily convert or permanently convert) the polyaxial pedicle screw arrangement in to a monoaxial-behaving screw.

In accordance with another and/or alternative non-limiting aspect of the present disclosure, there is provided a polyaxial pedicle screw arrangement wherein the pedicle screw can be configured for used in a variety of bones such as, but not limited to, spinal bones (e.g., cervical spine (C1-C7), thoracic spine (T1-T12), lumbar spine (L1-L5), sacral spine (S1-S5), tailbone). The pedicle screw includes a head portion 22 and a body portion 24. The head portion generally has a maximum cross-sectional area that is greater than a maximum cross-sectional area of the body portion. The head portion may or may not include external threads. The body portion generally includes threads; however, this is not required. The threads (when used) are generally spiral shaped; however, this is not required. For example, the body portion can be fully threaded, partially threaded, comprise a spiral or helical blade, and/or may comprise one or more tacks, deployable talons, expanding elements, or so forth. As can be appreciated, the body of the pedicle screw can be a peg or pin shape. The threads on the head portion and/or body portion of the screw (when used) are non-limiting (e.g., right-hand threads, left-hand threads, taper threads, “V” shape threads, metric threads, British threads, seller threads, square threads, acme threads, buttress threads, knuckle threads, worm threads, single and multi-threads). The end region of the body portion can optionally include a self-tapping or self-drilling tip; however, this is not required. The shape of the head portion is non-limiting. In one non-limiting embodiment, the cross-sectional shape of top region of the head portion is generally circular shaped; however, other shapes can be used. The head portion can optionally include a cavity to facilitate in inserting the orthopedic screw into a bone. The configuration of the cavity (when used) is non-limiting. Generally, the cavity is specially shaped to receive a tool to rotate and/or otherwise cause the pedicle screw to be inserted into a bone. Non-limiting shapes that can be used in the cavity include one or more dimples, ridges, bumps, textured areas, star shaped, polygonal shaped, or any other surface or shape. As can be appreciated, the cavity can optionally include a threaded inner surface.

In accordance with another and/or alternative non-limiting aspect of the present disclosure, there is provided a polyaxial pedicle screw arrangement wherein the tulip includes a body that includes a top portion and a bottom portion. The body generally has a circular cross-sectional shape; however, other cross-sectional shapes can be used (e.g., oval, triangular, square, rectangular, polygonal, etc.). In one non-limiting embodiment, the tulip is configured to facilitate in the connection to an orthopedic device OD (e.g., spinal rod, etc.). The outer surface of body can optionally include one or more surface structures (e.g., slot, cavity, rib, depression, etc.) that is used to facilitate in the medical instrument releasably engaging the outer surface of the body of tulip. The number, size, shape and configuration of the one or more surface structures are non-limiting. The top portion of the body includes of the tulip optionally includes one or more side guide surfaces that are configured to facilitate in the guiding and positioning of engagement legs of the inner body of the medical instrument during movement of the inner body relative to tulip. The top portion of the body includes a top cavity. The internal surface of the top cavity can include a connection surface such as, but not limited to, a threaded surface. The cross-sectional shape of the top cavity is generally circular; however, other shapes can be used. The longitudinal length of the top cavity is generally 50-100% (and all values and ranges therebetween) of the longitudinal length of the top portion. The top cavity includes a top opening that is configured to allow an instrument (e.g., screwdriver, locking tool, etc.) to be inserted into the top cavity of the top portion of the body to enable the instrument to access a locking device LD (e.g., locking screw, etc.) and manipulate (e.g., turn, push, move, etc.) the locking device LD. The side of the top portion includes first and second top side openings that are configured to receive an orthopedic device OD (e.g., rod, etc.). In one non-limiting embodiment, the top cavity includes a connection arrangement in the form of a threaded connection arrangement that is configured to receive a locking device LD (e.g., threaded lock screw, etc.) to secure an orthopedic device OD (e.g., rod, etc.) in top cavity. The first and second top side openings are configured to be positioned on opposite sides of the top cavity 40 and extend upwardly to the top opening of the top cavity 40 of the top portion. The first and second top side openings form two or more upwardly extending arms in the top portion 34. The longitudinal length of the first and second top side openings generally extends 10-100% (and all values and ranges therebetween) of the longitudinal length of the top portion. In one non-limiting arrangement, after an orthopedic device OD (e.g., rod, etc.) is inserted through and/or into the first and second top side openings, a locking device LD (e.g., threaded screw, etc. can be inserted into the top opening in the top cavity and connected thereto (e.g., the locking screw can be threaded on the threaded surface in the cavity of the top portion, etc.) to secure and lock the orthopedic device OD (e.g., rod, etc.) in position relative to the body of the tulip. As can be appreciated, many different arrangements can be used to secure the orthopedic device OD (e.g., rod, etc.) to the top portion of the body of the tulip. In another non-limiting embodiment, the first and second top side openings can optionally have a generally U-shaped configuration; however; other shapes can be used (e.g., triangular, square, rectangular, polygonal, oval, etc.). The size and shape of the first and second top side openings is generally the same; however, this is not required. The top portion can include first and second side slots that are configured to receive a portion of an upper pressure cap as will be discussed in more detail below. The bottom portion of the body generally has a longitudinal length that is 30-70% (and all values and ranges therebetween) of the longitudinal length of the top portion of the body; however, this is not required. The body generally includes a mid-opening that is positioned fully in the top portion, fully in the bottom portion, or partially in the top and bottom portion of the body of the tulip. The mid-opening is generally positioned about the central axis of the body of the tulip. The cross-sectional shape of the mid-opening is generally circular; however, this is not required. Generally, the longitudinal length of the mid-opening as measured along the longitudinal axis of the body is generally 5-40% (and all values and ranges therebetween) of the longitudinal length of the body. The maximum diameter of the mid-opening or the maximum cross-sectional area of the mid-opening is generally 50-90% (and all values and ranges therebetween) of the maximum diameter of the body or the maximum cross-sectional area of the body that contains the mid-opening. The bottom portion of the body includes a bottom cavity that is located below the mid-opening of the body. The size and/or shape of the bottom cavity of the bottom portion can be the same or different from the top cavity in the top portion; however, this is not required. In one non-limiting embodiment, the cross-sectional shape of the bottom cavity is generally circular; however, other shapes can be used. The longitudinal length of the bottom cavity is generally 50-100% (and all values and ranges therebetween) of the longitudinal length of the bottom portion. The internal surface of the bottom cavity can optionally include a connection surface (e.g., threaded surface, etc.) or be a smooth surface. The longitudinal length of the bottom cavity is generally 50-100% (and all values and ranges therebetween) of the longitudinal length of the bottom portion. The bottom cavity is shaped and sized to as to partially or fully telescopically receive the lower pressure cap/flexible collet as will be further discussed below.

In accordance with another and/or alternative non-limiting aspect of the present disclosure, there is provided a polyaxial pedicle screw arrangement wherein the upper pressure cap and the lower pressure cap/flexible cap are used to temporarily locked in place a polyaxial pedicle screw arrangement in position during a surgical procedure. The upper pressure cap is configured to be positioned above the lower pressure cap/flexible cap and is configured to engage the lower pressure cap/flexible cap. The upper pressure cap includes side flanges that are configured to slide into first and second side slots of top portion of tulip. When the side flanges are positioned in first and second side slots of top portion of tulip, the upper pressure cap is limited in the distance that the upper pressure cap can vertically move upwardly toward the top portion. Generally, when the side flanges are positioned first and second side slots of top portion of tulip, the upper pressure cap is prevented from vertically moving upwardly no more than 25% (e.g., 0.1-25% and all values and ranges therebetween) of the central axial length of the top portion of the tulip. The first and second side slots are configured to allow some upward and downward movement of the side flanges within first and second side slots to facilitate in the locking of the pedicle screw relative to the lower pressure cap/flexible cap as will be discussed in more device below. Each of side flanges includes one or more side slots (e.g., 1-4 side slots and all values and ranges therebetween) that are configured to receive a portion of lower pressure cap/flexible cap. The upper pressure cap include a central opening. Generally, the cross-sectional area or diameter of the central opening is less than the maximum cross-sectional area or maximum diameter of head portion of the pedicle screw such that the head portion is unable to fully pass through central opening. The central region of the upper pressure cap that is located between side flanges can optionally curve downwardly; however, this is not required. The upper pressure cap includes one or more engagement areas (e.g., 1-8 engagement areas and all values and ranges therebetween) that are configured to be engaged by a medical instrument. The one or more engagement area can be optionally located on at least a portion or all of the side flanges of the upper pressure cap. In one non-limiting arrangement, an engagement area is located on the top surface of each end region of each of side flanges. As will be discussed in more detail below, the upper pressure cap is configured to be engaged by a medical instrument at the one or more engagement areas, and pressure that is applied by the medical instrument on the one or more engagement areas causes movement of the lower pressure cap/flexible cap, that is located below the upper pressure cap, which in turn causes the polyaxial pedicle screw arrangement to be temporarily locked in in position. The one or more engagement areas can optionally include a recessed region that is configured to receive a portion of the medical instrument. In an alternative arrangement, the one or more engagement areas are in the form of openings that pass fully through the upper pressure cap. In such an arrangement, a portion of the medical instrument is configured to fully pass though the opening in one or more of the engagement areas and thereafter directly contact the lower pressure cap/flexible cap, that is located below the upper pressure cap, and cause movement of the lower pressure cap/flexible cap, which in turn causes the polyaxial pedicle screw arrangement to be temporarily locked in in position. As can be appreciated, a combination of these two arrangements or other alternative arrangement can be used to cause movement of the lower pressure cap/flexible cap by the medical instrument.

In accordance with another and/or alternative non-limiting aspect of the present disclosure, there is provided a polyaxial pedicle screw arrangement wherein the lower pressure cap/flexible cap includes a body that has a central axial cavity. The outer surface of body can have a circular cross-sectional shape; however, this is not required. Cavity generally has a circular cross-sectional shape. The cross-sectional area of cavity can be constant or can vary along the central axis of cavity. The size of cavity is selected such that the maximum diameter of maximum cross-sectional area of the head portion of the pedicle screw can pass through the bottom region of cavity. In one non-limiting arrangement, the size of cavity is selected such that the maximum diameter of maximum cross-sectional area of the head portion of the pedicle screw can pass through 30-100 % (and all values and ranges therebetween) of the longitudinal length of cavity. The top surface of body can optionally have a curved profile that partially recesses in body. The profile of top surface of body can be such that it closely mates with a portion of the profile of the bottom surface of the upper pressure cap; however, this is not required. One or more position flanges (e.g., 1-8 position flanges and all values and ranges therebetween) extend upwardly from the top surface of body. A portion or all of one of position flanges is configured to at least partially enter into one of side slots of side flanges of upper pressure cap when the polyaxial pedicle screw arrangement is fully assembled. The positioning of the position flanges into side slots of side flanges of upper pressure cap a) facilitates in proper positioning of the upper pressure cap relative to the lower pressure cap/flexible cap, and/or b) inhibits or prevents undesired movement of the lower pressure cap/flexible cap about the central axis of tulip (e.g., limits rotational movement of the lower pressure cap/flexible collet about a central axis of the tulip) when the polyaxial pedicle screw arrangement is fully assembled. Furthermore, the upper pressure cap inhibits or prevents upward movement of the lower pressure cap/flexible cap along the central axis of tulip when the polyaxial pedicle screw arrangement is fully assembled. The wall of the lower portion of body optionally includes one or more slots (e.g., 1-10 slots and all values and ranges therebetween) that extend from the base of the body to a point that is spaced from an upper edge of body. Generally, each of slots extends 40-95% (and all values and ranges therebetween) the longitudinal length of body. The slots can be optionally oriented on body such that adjacently positioned slots are equally spaced form one another; however, this is not required. The one or more slots are configured to enable the wall of the lower portion of body to be compressed and to reduce in cross-sectional area. In one non-limiting arrangement the cross-sectional area or diameter of the lower portion or bottom end of bottom cavity in body of tulip is less than a maximum cross-sectional area or maximum diameter of the bottom portion of body of lower pressure cap/flexible cap. Such reduction in cross-sectional area or diameter can be obtained by a) the bottom end portion of the bottom portion of body tapering inwardly, b) the changing of the thickness of the wall of the bottom portion, and/or c) the changing of the inner and/or outer diameter of the wall of the bottom portion. In one non-limiting arrangement, the thickness of the bottom portion of the body is varied such that the bottom end portion forms a bottom opening into bottom cavity that has a maximum cross-sectional area or maximum diameter that is less than a portion of the bottom cavity that is located above the bottom end portion. Due to the cross-sectional area or diameter of the lower portion or bottom end of bottom cavity in body of tulip being less than a maximum cross-sectional area or maximum diameter of the bottom portion of body of lower pressure cap/flexible cap, when the body of lower pressure cap/flexible cap is inserted into bottom cavity in body of tulip, the one or more slots enable the wall of the lower portion of body to be compressed and to reduce in cross-sectional area so as to pass through the lower portion or bottom end of bottom cavity in body of tulip. Once the bottom portion of body of lower pressure cap/flexible cap pass by the lower portion or bottom end of bottom cavity in body of tulip, the cross-sectional area or diameter of the bottom cavity increases thereby allowing the bottom portion of body of lower pressure cap/flexible cap to reexpand or spring or flex back to the original or near original (e.g., 90-99.999% of original and all values and ranges therebetween) cross-sectional area or diameter or position. Such reexpansion, spring back or flex back of the bottom portion of body of lower pressure cap/flexible cap inhibits or prevents that lower pressure cap/flexible cap from exiting through the lower portion or bottom end of bottom cavity in body of tulip without having to first applying a substantial force to the lower pressure cap/flexible cap to cause the lower portion of body to be compressed. The bottom region or end of body of lower pressure cap/flexible cap can optionally include a tapered base flange. The tapered base flange includes a tapered bottom region and optionally a tapered top region. The tapered top region, when used, facilitates in the insertion of the lower pressure cap/flexible cap into the bottom cavity in body of tulip during assembly of the polyaxial pedicle screw arrangement. The tapered bottom region is configured to engage the lower portion or bottom end of bottom cavity in body of tulip when the lower pressure cap/flexible cap is caused to move downwardly within the bottom cavity when a downwardly force from upper pressure cap is applied to the top surface of body of lower pressure cap/flexible cap. The upper pressure cap can be caused to produce such downward force on lower pressure cap/flexible cap when a medical instrument applies a downward force onto upper pressure cap as discussed above. When the polyaxial pedicle screw arrangement is fully assembled, the downward movement of the lower pressure cap/flexible cap causes the outer surface of the wall or base flange of body to engage the lower portion or bottom end of bottom cavity in body of tulip and thereby cause the cross-sectional area or diameter of the lower portion of body to be reduces, and such reduction in cross-sectional area or diameter of the lower portion of body causes the cross-sectional area or diameter of cavity to reduce and compress against the head portion of the pedicle screw and thereby prevent movement of the head portion of the pedicle screw within cavity. Such prevention of movement of the head portion of the pedicle screw within cavity causes the polyaxial pedicle screw arrangement to be temporarily locked in in position, thereby temporarily converting the polyaxial pedicle screw arrangement into a monoaxial-behaving pedicle screw arrangement.

In accordance with another and/or alternative non-limiting aspect of the present disclosure, there is provided a polyaxial pedicle screw arrangement wherein the downward force can be applied by one or more medical instruments, and wherein the downward force on the upper pressure cap causes the side flanges of upper pressure cap to move downwardly in first and second side slots of tulip. The size of the first and second side slots of tulip limit the distance of downward movement of upper pressure cap in the tulip. The downward movement of the upper pressure cap 60 causes the lower pressure cap/flexible cap to also move downwardly in tulip. As the lower pressure cap/flexible cap moves downwardly, the outer surface of the wall or base flange of body engages the lower portion or bottom end of bottom cavity in body of tulip and is cause to bend inwardly, which in turn causes the cross-sectional area or diameter of the lower portion of body to be reduced, and such reduction in cross-sectional area or diameter of the lower portion of body causes the cross-sectional area or diameter of cavity to reduce and compress against the head portion of the pedicle screw and thereby prevent movement of the head portion of the pedicle screw within cavity. Such prevention of movement of the head portion of the pedicle screw within cavity causes the polyaxial pedicle screw arrangement to be temporarily locked in in position, thereby temporarily converting the polyaxial pedicle screw arrangement into a monoaxial-behaving pedicle screw arrangement. As can be appreciated, when the downward force is removed from the upper pressure cap, the upper pressure cap can move upwardly in first and second side slots of tulip, thus also allowing lower pressure cap/flexible cap to also move upwardly in tulip, which upward movement of lower pressure cap/flexible cap allow the walls of the lower portion of body to expand and once again allow movement of the head portion of the pedicle screw 20 relative to the lower pressure cap/flexible cap.

In accordance with another and/or alternative non-limiting aspect of the present disclosure, there is provided a polyaxial pedicle screw arrangement wherein the pedicle screw is inserted into the tulip when the lower pressure cap/flexible cap has also been inserted into the tulip. When the lower pressure cap/flexible cap is positioned in its upper portion, the head portion of the pedicle screw is able to be inserted into cavity of body of the lower pressure cap/flexible cap. In such position, the portion of the body and the tapered base flange that are adjacent to one or more slots can flex outwardly a sufficient distance within bottom cavity of tulip to enable the head portion of the pedicle screw to fully pass by tapered base flange and into cavity of body of the lower pressure cap/flexible cap. Once the head portion of the pedicle screw passes by tapered base flange, the portion of the body and the tapered base flange that are adjacent to one or more slots can flex back to their original positions. The bottom cavity of tulip can be configured such that only when the lower pressure cap/flexible cap is positioned in its upper portion can the body and the tapered base flange that are adjacent to one or more slots flex outwardly a sufficient distance within bottom cavity of tulip to enable the head portion of the pedicle screw to fully pass by tapered base flange and into cavity of body of the lower pressure cap/flexible cap.

In accordance with another and/or alternative non-limiting aspect of the present disclosure, there is provided a polyaxial pedicle screw arrangement wherein the medical instrument includes an outer body and an inner body. The inner body is configured to be rotatably positioned in the outer body and rotatable about a central longitudinal axis of the medical instrument. The top portion of the inner body extends upwardly from a top portion of the outer body. The top portion of the inner body optionally includes one or more engagement members that are configured to be engaged by a medical tool (not shown) to facilitate in the rotation of the inner body relative to the outer body of the medical instrument. The size, shape and configuration of the one or more engagement members are non-limiting. The top portion of the inner body has a non-limiting hexagonal cross-sectional shape with six flat faced engagement members. The bottom portion of outer body is configured to releasably engage the outer surface of tulip. The outer surface of the outer body can optionally include one or more gripping regions that can be used by a user to facilitate in the gripping of the medical instrument during use. The size, shape and configuration of the one or more gripping regions are non-limiting. The outer body can optionally include one or more side openings that can be used to a) reduce the weight of the outer body, and/or b) enable a user to view the movement of one or more portions of the inner body within the outer body. The size, shape and/or configuration of the one or more side openings are non-limiting. The outer body can optionally include one or more upper side openings that can be used to a) reduce the weight of the outer body, and/or b) enable a user to view the distance of longitudinal movement of the inner body within outer body. The size, shape and/or configuration of the one or more upper side openings are non-limiting. The inner body can optionally include numbering or other types of designations that inform a user the distance of longitudinal movement of the inner body within outer body during use of the medical device. The outer body can optionally include one or more bottom slots or openings that can be used to a) reduce the weight of the outer body, b) enable a used to view the movement of one or more portions of the inner body within the outer body, and/or c) enable a user to view the positioning of the engagement legs 200 relative to the engagement areas of the upper pressure cap. The size, shape and/or configuration of the one or more bottom slots or openings are non-limiting. A top portion of the outer body optionally includes one or more gripping members that are configured to be engaged by a medical tool (not shown) to a) maintain the position of the outer body during a surgical procedure, b) rotate the outer body during a surgical procedure, and/or c) maintain the position of the outer body during rotation of the top portion of the inner body. The size, shape and configuration of the one or more gripping members are non-limiting.

In accordance with another and/or alternative non-limiting aspect of the present disclosure, there is provided a polyaxial pedicle screw arrangement wherein the top portion of the inner body optionally includes one or more engagement members that are configured to be engaged by a medical tool (not shown) to facilitate in the rotation of the inner body relative to the outer body of the medical instrument. The top portion of the inner body is illustrated as having a non-limiting hexagonal cross-sectional shape with six flat faced engagement members; however, it will be appreciated that other configurations of the top portion can be used. Positioned below the top portion is a threaded region. The threaded region is configured to engage a threaded surface on the top portion of the outer body of the medical device. When the inner body is rotated relative to the outer body about the central axis of the medical instrument, the threaded region on the inner body and the threaded surface on the top portion of the outer body cause the inner body to move upwardly and downwardly relative to the outer body depending on the direction of rotation of the inner body. As the inner body moves upwardly and/or downwardly relative to the outer body, a user can view the degree of movement of the inner body by viewing the numbering or other types of designations (e.g., A, B, C; 1, 2, 3, etc.) on the top portion of the inner body via upper side openings in the outer body. The bottom region of the top portion includes a coupling arrangement that is configured to secure the top portion of the inner body to the bottom portion of the inner body. The bottom region of the bottom portion includes a plurality of engagement legs. The bottom portion can optionally include one or more extension sections that are configured to be coupled together and used to obtain the desired length of the inner body. The one or more extension sections can optionally include one or more slots. The one or more slots can be used to reduce the weight of the inner body and/or reduce the amount of material used in the inner body. The inner body can include two extension sections. The first extension section is connected at one end to the top portion of the inner body and the other end of the first top extension section is connected to the top end of the second extension section The other end of the second extension section is connected to the bottom region or bottom section of the inner body. The arrangement used to couple the extension sections together and/or to other portions of the inner body is non-limiting (e.g., threaded connection, slot & lock connection, adhesive, etc.).

In accordance with another and/or alternative non-limiting aspect of the present disclosure, there is provided a polyaxial pedicle screw arrangement wherein when an orthopedic device OD (e.g., rod, etc.) is inserted into first and second top side openings of cavity in the top portion of tulip and thereafter secured in cavity by a locking device LD (e.g., threaded lock screw, etc.) or other type of connector that is connected to connection arrangement, the orthopedic device is caused to press downwardly and cause a downward force to be applied on upper pressure cap Such downward force on the upper pressure cap causes the lower pressure cap/flexible cap to also move downwardly in tulip. As the lower pressure cap/flexible cap moves downwardly, the outer surface of the wall or base flange of body engages the lower portion or bottom end of bottom cavity in body of tulip and is cause to bend inwardly, which in turn causes the cross-sectional area or diameter of the lower portion of body to be reduced, and such reduction in cross-sectional area or diameter of the lower portion of body causes the cross-sectional area or diameter of cavity to reduce and compress against the head portion of the pedicle screw and thereby prevent movement of the head portion of the pedicle screw within cavity. Such prevention of movement of the head portion of the pedicle screw within cavity causes the polyaxial pedicle screw arrangement to be locked in in position, thereby converting the polyaxial pedicle screw arrangement into a monoaxial-behaving pedicle screw arrangement.

In accordance with another and/or alternative non-limiting aspect of the present disclosure, there is provided a polyaxial pedicle screw arrangement wherein one or more of the components can be formed of a variety of materials. In one non-limiting embodiment, the metal portion of the polyaxial pedicle screw arrangement is partially (e.g. 1-99.999 wt. % and all values and ranges therebetween) or fully formed of a metal material that includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, or m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) rhenium (e.g., 5-99 awt. % rhenium and all values and ranges therebetween). As used herein, atomic weight percent (awt. %) or atomic percentage (awt%) or atomic percent (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. It has been found that for several metal alloys the inclusion of at least 15 awt. % rhenium results in the ductility and/or tensile strength of the metal alloy to improve as compared to a metal alloy is that absent rhenium. Such improvement in ductility and/or tensile strength due to the inclusion of at least 15 awt. % rhenium in the metal alloy is referred to as the “rhenium effect.” As defined herein, a “rhenium effect” is a) an increase of at least 10% in ductility of the metal alloy caused by the addition of rhenium to the metal alloy, and/or b) an increase of at least 10% in tensile strength of the metal alloy caused by the addition of rhenium to the metal alloy. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In another and/or alternative non-limiting aspect of the disclosure, the metal portion of the polyaxial pedicle screw arrangement that is partially or fully formed of a metal material includes stainless steel, CoCr alloys, TiAlV alloys, aluminum alloys, nickel alloys, titanium alloys, tungsten alloys, molybdenum alloys, copper alloys, MP35N alloys, or beryllium-copper alloys that have been modified to include at least 15 awt. % rhenium so as to result in improved ductility and/or tensile strength as compared to the same metal alloy that is absent rhenium. As defined herein, a stainless-steel alloy (SS alloy) includes at least 50 wt. % (weight percent) iron, 10-28 wt. % chromium, 0-35 wt. % nickel, and optionally one or more of 0-4 wt. % molybdenum, 0-2 wt. % manganese, 0-0.75 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, and 0-2 wt. % tungsten. A 316L alloy that falls within a stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a cobalt-chromium alloy (CoCr alloy) includes 30-68 wt. % cobalt, 15-32 wt. % chromium, and optionally one or more of 1-38 wt. % nickel, 2-18 wt. % molybdenum, 0-18 wt. % iron, 0-1 wt. % titanium, 0-0.15 wt. % manganese, 0-0.15 wt. % silver, 0-0.25 wt. % carbon, 0-16 wt. % tungsten, 0-2 wt. % silicon, 0-2 wt. % aluminum, 0-1 wt. % iron, 0-0.1 wt. % boron, 0-0.15 wt. % silver, and 0-2 wt. % titanium. As a MP35N alloy that falls within a CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a Phynox and Elgiloy alloy that falls within a CoCr alloy includes 38-42 wt. % cobalt, 18-22 wt. % chromium, 14-18 wt. % iron, 13-17 wt. % nickel, 6-8 wt. % molybdenum. As defined herein, a L605 alloy that falls within a CoCr alloy includes 18-22 wt. % chromium, 14-16 wt. % tungsten, 9-11 wt. % nickel, balance cobalt. As defined herein, a titanium-aluminum-vanadium alloy (TiAlV alloy) includes 4-8 wt. % aluminum, 3-6 wt. % vanadium, 80-93 wt. % titanium, and optionally one or more of 0-0.4 wt. % iron, 0-0.2 wt. % carbon, 0-0.5 wt. % yttrium. A Ti-6Al-4V alloy that falls with a TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, an aluminum alloy includes 80-99 wt. % aluminum, and optionally one or more 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a nickel alloy includes 30-98 wt. % nickel, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a titanium alloy includes 80-99 wt. % titanium, and optionally one of more of 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a tungsten alloy includes 85-98 wt. % tungsten, and optionally one or more of 0-8 wt. % nickel, 0-5 wt. % copper, 0-5 wt. % molybdenum, 0-4 wt. % iron. As defined herein, a molybdenum alloy includes 90-99.5 wt. % molybdenum, and optionally one or more of 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a copper alloy includes 55-95 wt. % copper, and optionally one or more of 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a beryllium-copper alloy includes 95-98.5 wt. % copper, 1-4 wt. % beryllium, and optionally one or more of 0-1 wt. % cobalt, and 0-0.5 wt. % silicon. As defined herein, a titanium-nickel alloy (e.g., Nitinol alloy) includes 42-58 wt. % nickel and 42-58 wt. % titanium.

In accordance with another and/or alternative non-limiting aspect of the present disclosure, the metal portion of the polyaxial pedicle screw arrangement that is partially or fully formed of a metal material includes a metal alloy that contains at least 15 awt. % rhenium. It has been found that for several metal alloys the inclusion of at least 15 awt%. It has been found for some metal alloys (e.g., stainless steel, CoCr alloys, TiAlV alloys, aluminum alloys, nickel alloys, titanium alloys, tungsten alloys, molybdenum alloys, copper alloys, MP35N alloys, beryllium-copper alloys, etc.), the inclusion of at least 15 awt. % rhenium results in improved ductility and/or tensile strength. It has been found that the addition of rhenium to a metal alloy can result in the formation of a twining alloy in the metal alloy that results in the overall ductility of the metal alloy to increase as the yield and tensile strength increases as a result of reduction and/or work hardening of the metal alloy that includes the rhenium addition. The rhenium effect has been found to occur when the atomic weight of rhenium in the metal alloy is at least 15 awt. % (e.g., 15-99 awt. % rhenium in the metal alloy and all values and ranges therebetween). For example, for stainless-steel alloys, the rhenium effect can begin to be present when the stainless-steel alloy is modified to include a rhenium amount of at least 5-10 wt. % (and all values and ranges therebetween) of the stainless-steel alloy. For CoCr alloys, the rhenium effect can begin to be present when the CoCr alloy is modified to include a rhenium amount of at least 4.8-9.5 wt. % (and all values and ranges therebetween) of the CoCr alloy. For TiAlV alloys, the rhenium effect can begin to be present when the TiAlV alloy is modified to include a rhenium amount of at least 4.5-9 wt. % (and all values and ranges therebetween) of the TiAlV alloy. It can be appreciated, the rhenium content in the above non-limiting examples can be greater than the minimum amount to create the rhenium effect in the metal alloy.

In accordance with another and/or alternative aspect of the present disclosure, the metal portion of the polyaxial pedicle screw arrangement that is partially or fully formed of a metal material includes at least 5 awt. % (e.g., 5-99 awt. % and all values and ranges therebetween) rhenium, and 0.1-96 wt. % (and all values and ranges therebetween) of one or more additives selected from the group of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a rhenium effect. In one non-limiting embodiment, the metal portion of the polyaxial pedicle screw arrangement that is partially or fully formed of a metal material is a stainless-steel alloy that has been modified to include at least 15 awt. % rhenium. In another non-limiting embodiment, the metal portion of the polyaxial pedicle screw arrangement that is partially or fully formed of a metal material is a cobalt chromium alloy that has been modified to include at least 15 awt. % rhenium. In another non-limiting embodiment, the metal portion of the polyaxial pedicle screw arrangement that is partially or fully formed of a metal material is a TiAlV alloy that has been modified to include at least 15 awt. % rhenium. In another non-limiting embodiment, the metal portion of the polyaxial pedicle screw arrangement that is partially or fully formed of a metal material is an aluminum alloy that has been modified to include at least 15 awt. % rhenium. In another non-limiting embodiment, the metal portion of the polyaxial pedicle screw arrangement that is partially or fully formed of a metal material is a nickel alloy that has been modified to include at least 15 awt. % rhenium. In another non-limiting embodiment, the metal portion of the polyaxial pedicle screw arrangement that is partially or fully formed of a metal material is a titanium alloy that has been modified to include at least 15 awt. % rhenium. In another non-limiting embodiment, the metal portion of the polyaxial pedicle screw arrangement that is partially or fully formed of a metal material is a tungsten alloy that has been modified to include at least 15 awt. % rhenium. In another non-limiting embodiment, the metal portion of the polyaxial pedicle screw arrangement that is partially or fully formed of a metal material is a molybdenum alloy that has been modified to include at least 15 awt. % rhenium. In another non-limiting embodiment, the metal portion of the polyaxial pedicle screw arrangement that is partially or fully formed of a metal material is a copper alloy that has been modified to include at least 15 awt. % rhenium. In another non-limiting embodiment, the metal portion of the polyaxial pedicle screw arrangement that is partially or fully formed of a metal material is a beryllium-copper alloy that has been modified to include at least 15 awt. % rhenium.

In accordance with another and/or alternative aspect of the present disclosure, the metal portion of the polyaxial pedicle screw arrangement that is partially or fully formed of a metal material includes rhenium and molybdenum, and the weight percent of rhenium in the metal alloy is optionally greater than the weight percent of molybdenum in the metal alloy, and the weight percent of one or more additive (e.g., aluminum, boron, beryllium, bismuth, cadmium, calcium, cerium, chromium, cobalt, copper, gallium, gold, hafnium, iridium, iron, lanthanum, lanthanum oxide, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium) in the metal alloy is optionally greater that the weight percent of molybdenum in the metal alloy, and the metal alloy optionally includes 0-2 wt. % of a combination of other components other than the additives (e.g., carbon, oxygen, phosphorous, sulfur, hydrogen, lead, nitrogen, etc.). In one non-limiting embodiment, the metal portion of the polyaxial pedicle screw arrangement that is partially or fully formed of a metal material includes rhenium and molybdenum, and the weight percent of rhenium plus the combined weight percent of additives is greater than the weight percent of molybdenum, and the metal alloy optionally includes 0-2 wt. % of a combination of other components other than the additives (e.g., carbon, oxygen, phosphorous, sulfur, hydrogen, lead, nitrogen, etc.).

In accordance with another and/or alternative aspect of the present disclosure, the metal portion of the polyaxial pedicle screw arrangement that is partially or fully formed of a metal material includes rhenium and molybdenum, and the atomic weight percent of rhenium to the atomic weight percent of the combination of one or more of bismuth, niobium, tantalum, tungsten, titanium, vanadium, chromium, manganese, yttrium, zirconium, technetium, ruthenium, rhodium, hafnium, osmium, copper, and iridium is 0.4:1 to 2.5:1 (and all values and ranges therebetween).

In accordance with another and/or alternative aspect of the present disclosure, the metal portion of the polyaxial pedicle screw arrangement that is partially or fully formed of a metal material includes at least 5 awt. % (e.g., 5-99 awt. % and all values and ranges therebetween) rhenium plus at least two metals selected from the group of molybdenum, bismuth, chromium, iridium, niobium, tantalum, titanium, yttrium, and zirconium, and the content of the metal alloy that includes other elements and compounds is 0-0.1 wt. %. In another non-limiting embodiment, the metal alloy includes rhenium, molybdenum, and chromium. In another non-limiting embodiment, the metal alloy includes at least 35 wt. % (e.g., 35-75 wt. % and all values and ranges therebetween) rhenium, and the metal alloy also includes chromium. In one non-limiting embodiment, the metal alloy includes at least 35 wt. % rhenium and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more of aluminum, boron, beryllium, bismuth, cadmium, calcium, cerium, chromium, cobalt, copper, gallium, gold, hafnium, iridium, iron, lanthanum, lanthanum oxide, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen. In another non-limiting embodiment, the metal alloy includes 15-50 awt. % rhenium (and all values and ranges therebetween) and 0.5-70 awt. % chromium (and all values and ranges therebetween). In another non-limiting embodiment, the metal alloy includes 15-50 awt. % rhenium (and all values and ranges therebetween) and 0.5-70 awt. % tantalum (and all values and ranges therebetween). In another non-limiting embodiment, the metal alloy includes 15-50 awt. % rhenium (and all values and ranges therebetween) and 0.5-70 awt. % niobium (and all values and ranges therebetween). In another non-limiting embodiment, the metal alloy includes 15-50 awt. % rhenium (and all values and ranges therebetween) and 0.5-70 awt. % titanium (and all values and ranges therebetween). In another non-limiting embodiment, the metal alloy includes 15-50 awt. % rhenium (and all values and ranges therebetween) and 0.5-70 awt. % zirconium (and all values and ranges therebetween). In another non-limiting embodiment, the metal alloy includes 15-50 awt. % rhenium (and all values and ranges therebetween) and 0.5-70 awt. % molybdenum (and all values and ranges therebetween). In another non-limiting embodiment, the metal alloy includes at least 15 awt. % rhenium, greater than 50 wt. % titanium (e.g., 51-80 wt. % and all values and ranges therebetween), 15-45 wt. % (and all values and ranges therebetween) niobium, 0-10 wt. % (and all values and ranges therebetween) zirconium, 0-15 wt. % (and all values and ranges therebetween) tantalum, and 0-8 wt. % molybdenum (and all values and ranges therebetween).

In accordance with another and/or alternative aspect of the present disclosure, the metal alloy that is used to partially or fully form the polyaxial pedicle screw arrangement is partially for fully formed of a refractory metal alloy, and wherein the refractory metal alloy includes at least 20 wt. % of one or more of niobium, tantalum or tungsten, and wherein the refractory metal alloy includes 0-30 wt. % molybdenum (and all values and ranges therebetween), and wherein the refractory metal alloy includes at least 5 awt. % rhenium (e.g., 5-80 awt. % rhenium and all values and ranges therebetween).

In accordance with another and/or alternative aspect of the present disclosure, the metal alloy that is used to partially or fully form the polyaxial pedicle screw arrangement is partially for fully formed of a metal alloy includes at least 5 awt. % rhenium (e.g., 5-99 awt. % rhenium and all values and ranges therebetween), and at least 0.1 wt. % of one or more additive metals selected from aluminum, bismuth, chromium, cobalt, copper, hafnium, iridium, iron, magnesium, manganese, nickel, niobium, osmium, rhodium, ruthenium, silicon, silver, tantalum, technetium, titanium, tungsten, vanadium, yttrium, and zirconium, and wherein the metal alloy includes 0-30 wt. % molybdenum (and all values and ranges therebetween), and wherein a combined weight percent of rhenium and the additive metals is 70-100 wt. % (and all values and ranges therebetween).

In accordance with another and/or alternative aspect of the present disclosure, the metal alloy that is used to partially or fully form the polyaxial pedicle screw arrangement is partially for fully formed of a metal alloy of stainless steel that has been modified with at least 5 awt. % rhenium (e.g., 5-50 awt. % rhenium and all values and ranges therebetween), and wherein a combined weight percent of iron, chromium, nickel, tantalum, niobium, copper, manganese, aluminum, titanium, selenium, vanadium, tungsten and rhenium is 70-100 wt. % (and all values and ranges therebetween).

In accordance with another and/or alternative aspect of the present disclosure, the metal alloy that is used to partially or fully form the polyaxial pedicle screw arrangement is partially for fully formed of a metal alloy of cobalt-chromium alloy that has been modified with at least 5 awt. % rhenium (e.g., 5-50 awt. % rhenium and all values and ranges therebetween), and wherein a combined weight percent of cobalt, chromium, nickel, iron, titanium, manganese, silver, tungsten, silicon, aluminum, iron, boron, silver, titanium, and rhenium is 70-100 wt. % (and all values and ranges therebetween).

In accordance with another and/or alternative aspect of the present disclosure, the metal alloy that is used to partially or fully form the polyaxial pedicle screw arrangement is partially for fully formed of a metal alloy of titanium-aluminum-vanadium alloy that has been modified with at least 5 awt. % rhenium (e.g., 5-50 awt. % rhenium and all values and ranges therebetween), and wherein a combined weight percent of aluminum, vanadium, titanium, iron, yttrium and rhenium is 70-100 wt. % (and all values and ranges therebetween).

In accordance with another and/or alternative aspect of the present disclosure, the metal alloy that is used to partially or fully form the polyaxial pedicle screw arrangement is partially for fully formed of a metal alloy of aluminum alloy that has been modified with at least 5 awt. % rhenium (e.g., 5-50 awt. % rhenium and all values and ranges therebetween), and wherein a combined weight percent of aluminum, silicon, magnesium, manganese, scandium, beryllium, yttrium, cerium, chromium, iron, zinc, titanium, lithium, silver, calcium, zirconium, cadmium, bismuth, nickel, vanadium, gallium, copper, and rhenium is 70-100 wt. % (and all values and ranges therebetween).

In accordance with another and/or alternative aspect of the present disclosure, the metal alloy that is used to partially or fully form the polyaxial pedicle screw arrangement is partially for fully formed of a metal alloy of nickel alloy that has been modified with at least 5 awt. % rhenium (e.g., 5-50 awt. % rhenium and all values and ranges therebetween), and wherein a combined weight percent of nickel, chromium, iron, copper, cobalt, aluminum, tantalum, tungsten, titanium, niobium, silicon, and rhenium is 70-100 wt. % (and all values and ranges therebetween).

In accordance with another and/or alternative aspect of the present disclosure, the metal alloy that is used to partially or fully form the polyaxial pedicle screw arrangement is partially for fully formed of a metal alloy of titanium alloy that has been modified with at least 5 awt. % rhenium (e.g., 5-50 awt. % rhenium and all values and ranges therebetween), and wherein a combined weight percent of titanium, aluminum, tin, palladium, vanadium, nickel, ruthenium, chromium, zirconium, niobium, silicon, cobalt, iron, and rhenium is 70-100 wt. % (and all values and ranges therebetween).

In accordance with another and/or alternative aspect of the present disclosure, the metal alloy that is used to partially or fully form the polyaxial pedicle screw arrangement is partially for fully formed of a metal alloy of tungsten alloy that has been modified with at least 5 awt. % rhenium (e.g., 5-50 awt. % rhenium and all values and ranges therebetween), and wherein a combined weight percent of tungsten, nickel, copper, iron, and rhenium is 70-100 wt. % (and all values and ranges therebetween).

In accordance with another and/or alternative aspect of the present disclosure, the metal alloy that is used to partially or fully form the polyaxial pedicle screw arrangement is partially for fully formed of a metal alloy of copper alloy that has been modified with at least 5 awt. % rhenium (e.g., 5-50 awt. % rhenium and all values and ranges therebetween), and wherein a combined weight percent of copper, zinc, tin, iron, silicon, manganese, aluminum, beryllium, cobalt, nickel, and rhenium is 70-100 wt. % (and all values and ranges therebetween).

In accordance with another and/or alternative aspect of the present disclosure, the metal alloy that is used to partially or fully form the polyaxial pedicle screw arrangement is partially for fully formed of a metal alloy of beryllium-copper alloy that has been modified with at least 5 awt. % rhenium (e.g., 5-50 awt. % rhenium and all values and ranges therebetween), and wherein a combined weight percent of copper, beryllium, cobalt, silicon, and rhenium is 70-100 wt. % (and all values and ranges therebetween).

In accordance with another and/or alternative aspect of the present disclosure, the metal alloy that is used to partially or fully form the polyaxial pedicle screw arrangement is partially for fully formed of a metal alloy of titanium-nickel alloy that has been modified with at least 5 awt. % rhenium (e.g., 5-50 awt. % rhenium and all values and ranges therebetween), and wherein a combined weight percent of nickel, titanium, and rhenium is 70-100 wt. % (and all values and ranges therebetween).

In another and/or alternative non-limiting aspect of the present disclosure, the metal alloy used to partially or fully form the polyaxial pedicle screw arrangement can be nitrided; however, this is not required. The thickness of the nitrided surface layer is less than about 1 mm. In one non-limiting embodiment, the thickness of the nitrided surface layer is at least about 50 nm and less than about 1 mm (and all values and ranges therebetween). In another non-limiting embodiment, the thickness of the nitrided surface layer is at least about 50 nm and less than about 0.1 mm. When a MoRe alloy is nitrided, the weight percent of the nitrogen in the nitrided surface layer is less than a weight percent of the molybdenum in the nitrided surface layer. Also, the weight percent of nitrogen in the nitrided surface layer is less than a weight percent of the rhenium in the nitrided surface layer. In one non-limiting composition of the nitrided surface layer on a MoRe alloy (e.g., 40-99 wt. % molybdenum, 1-40 wt. % rhenium), the nitride surface layer comprises 40-99 wt. % molybdenum (and all values and ranges therebetween), 1-40 wt. % rhenium (and all values and ranges therebetween), and 0.0001-5 wt. % nitrogen (and all values and ranges therebetween). In another non-limiting composition of the nitrided surface layer, the nitrided surface layer comprises 40-99 wt. % molybdenum, 1-40 wt. % rhenium, and 0.001-1 wt. % nitrogen. The nitriding process can be used to increase surface hardness and/or wear resistance of the polyaxial pedicle screw arrangement, and/or inhibits or prevents discoloration of the refractory metal alloy (e.g., discoloration by oxidation, etc.). For example, the nitriding process increases the wear resistance of articulation surfaces or surfaces wear on the refractory metal alloy used in the polyaxial pedicle screw arrangement to extend the life of the polyaxial pedicle screw arrangement, increases the wear life of mating surfaces on the polyaxial pedicle screw arrangement, and/or reduces particulate generation from use of the polyaxial pedicle screw arrangement and/or maintains the outer surface appearance of the metal alloy on the polyaxial pedicle screw arrangement.

In yet another and/or alternative non-limiting aspect of the present disclosure, the polyaxial pedicle screw arrangement can include, contain, and/or be coated with one or more agents that facilitate in the success of the polyaxial pedicle screw arrangement and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. The type and/or amount of an agent included in a polyaxial pedicle screw arrangement and/or coated on polyaxial pedicle screw arrangement can vary. When two or more agents are included in and/or coated on polyaxial pedicle screw arrangement, the amount of two or more agents can be the same or different. The type and/or amount of agent included on, in and/or in conjunction with polyaxial pedicle screw arrangement are generally selected to address one or more clinical events. Typically, the amount of agent included on, in, and/or used in conjunction with the polyaxial pedicle screw arrangement is about 0.01-100 μg per mm² and/or at least about 0.00001 wt. % of the device; however, other amounts can be used. In one non-limiting embodiment of the disclosure, the polyaxial pedicle screw arrangement can be partially or fully coated and/or impregnated with one or more agents to facilitate in the success of a particular medical procedure.

In a further and/or alternative non-limiting aspect of the present disclosure, the one or more agents on and/or in the polyaxial pedicle screw arrangement (when used) can be released in a controlled manner to provide the area in question to be treated with the desired dosage of agent over a sustained period of time. The polyaxial pedicle screw arrangement can be designed such that 1) all the agent on and/or in the polyaxial pedicle screw arrangement is controllably released, 2) some of the agent on and/or in the polyaxial pedicle screw arrangement is controllably released and some of the agent on the polyaxial pedicle screw arrangement is non-controllably released, or 3) none of the agent on and/or in the polyaxial pedicle screw arrangement is controllably released. The polyaxial pedicle screw arrangement can also be designed such that the rate of release of the one or more agents from the polyaxial pedicle screw arrangement is the same or different. The polyaxial pedicle screw arrangement can also be designed such that the rate of release of the one or more agents from one or more regions on the polyaxial pedicle screw arrangement is the same or different. Non-limiting arrangements that can be used to control the release of one or more agents from the polyaxial pedicle screw arrangement include 1) at least partially coating one or more agents with one or more polymers, 2) at least partially incorporating and/or at least partially encapsulating one or more agents into and/or with one or more polymers, and/or 3) inserting one or more agents in pores, passageway, cavities, etc., in the polyaxial pedicle screw arrangement and at least partially coating or covering such pores, passageway, cavities, etc., with one or more polymers. As can be appreciated, other or additional arrangements can be used to control the release of one or more agents from the polyaxial pedicle screw arrangement. The thickness of each polymer layer and/or layer of agent is generally at least about 0.01 μm and is generally less than about 150 μm (e.g., 0.01-149.9999 μm and all values and ranges therebetween). In one non-limiting embodiment, the thickness of a polymer layer and/or layer of agent is about 0.02-75μm, more particularly about 0.05-50 μm, and even more particularly about 1-30 μm.

In yet another and/or alternative non-limiting aspect of the disclosure, the polyaxial pedicle screw arrangement can include a marker material. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.), and/or other types of electromagnetic waves (e.g., microwaves, visible light, infrared waves, ultraviolet waves, etc.). In one non-limiting embodiment, the marker material is visible to x-rays (i.e., radiopaque). The marker material can form all or a portion of the polyaxial pedicle screw arrangement and/or be coated on one or more portions (flaring portion and/or body portion, at ends of polyaxial pedicle screw arrangement, at or near transition of body portion and flaring section, etc.) of the polyaxial pedicle screw arrangement. The location of the marker material can be on one or multiple locations on the polyaxial pedicle screw arrangement. The size of the one or more regions that include the marker material can be the same or different.

In a further and/or alternative non-limiting aspect of the present disclosure, the polyaxial pedicle screw arrangement or one or more regions of the polyaxial pedicle screw arrangement can be constructed by use of one or more microelectromechanical manufacturing (MEMS) techniques (e.g., micro-machining, laser micro-machining, laser micro-machining, micro-molding, etc.); however, other or additional manufacturing techniques can be used.

In still yet another and/or alternative non-limiting aspect of the present disclosure, there is provided a near net process for a body or other metal component of the polyaxial pedicle screw arrangement. In one non-limiting embodiment of the disclosure, there is provided a method of powder pressing materials and increasing the strength post sintering by imparting additional cold work. In one non-limiting embodiment, the green part is pressed and then sintered. Thereafter, the sintered part is again pressed to increase its mechanical strength by imparting cold work into the pressed and sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered and re-pressed) meets the dimensional requirements of the final formed part. For a Mo47.5Re alloy, MoRe alloy, ReW alloy, molybdenum alloy, tungsten alloy, rhenium alloy, other type of refractory metal alloy, or TWIP alloy formed of a high titanium content, a prepress pressure of 1-300 tsi (1 ton per square inch) (and all values and ranges therebetween) can be used followed by a sintering process of at least 1600° C. (e.g., 1600-2600° C. and all values and ranges therebetween) and a post sintering press at a pressure of 1-300 tsi (and all values and ranges therebetween) at a temperature of at least 20° C. (e.g., 20-100° C. and all values and ranges therebetween; 20-40° C., etc.). There is also provided a process of increasing the mechanical strength of a pressed metal part by repressing the post-sintered part to add additional cold work into the material, thereby increasing its mechanical strength. There is also provided a process of powder pressing to a near net or final part using metal powder. In one non-limiting embodiment, the metal powder used to form the near net or final part includes a minimum of 40% rhenium by weight and at least 30% molybdenum, and the remainder can optionally include one or more elements of tungsten, tantalum, zirconium, iridium, titanium, bismuth, and yttrium. In another non-limiting embodiment, the metal powder used to form the near net or final part includes 20-80 wt. % rhenium (and all values and ranges therebetween), 20-80 wt. % molybdenum (and all values and ranges therebetween), and optionally one or more elements of tungsten, tantalum, zirconium, iridium, titanium, bismuth, and yttrium. In another non-limiting embodiment, the metal powder used to form the near net or final part includes tungsten (20-60 wt. % and all values and ranges therebetween), rhenium (20-80 wt. % and all values and ranges therebetween) and one or more other elements 0-5 wt. % (and all values and ranges therebetween). In another non-limiting embodiment, the metal powder used to form the near net or final part includes tungsten (20-80 wt. % and all values and ranges therebetween), rhenium (20-80 wt. % and all values and ranges therebetween), molybdenum (0-15 wt. % and all values and ranges therebetween), and one or more other elements 0-5 wt. % (and all values and ranges therebetween). In another non-limiting embodiment, the metal powder used to form the near net or final part includes tungsten (20-80 wt. % and all values and ranges therebetween), copper (1-30 wt. % and all values and ranges therebetween), and one or more other elements 0-5 wt. % (and all values and ranges therebetween). In another non-limiting embodiment, the metal powder used to form the near net or final part includes a titanium alloy or a cobalt alloy. The ductility of the refractory metal alloy measured as % reduction in area can increase the yield and ultimate tensile strength can increase.

In accordance with another and/or alternative aspect of the present disclosure, there is optionally provided a near net process for one or more portions of the polyaxial pedicle screw arrangement. In one non-limiting embodiment of the disclosure, there is provided a method of powder pressing materials and increasing the strength post-sintering by imparting additional cold work. In one non-limiting embodiment, the green part is pressed and then sintered. Thereafter, the sintered part is again pressed to increase its mechanical strength by imparting cold work into the pressed and sintered part.

In accordance with another and/or alternative aspect of the present disclosure, the metal alloy that is used to partially or fully form the metal portion of the polyaxial pedicle screw arrangement can optionally be initially formed into a blank, a rod, a tube, etc., and then finished into final form by one or more finishing processes. The metal alloy blank, rod, tube, etc., can be formed by various techniques such as, but not limited to, 1) melting the metal alloy and/or metals that form the metal alloy (e.g., vacuum arc melting, etc.) and then extruding and/or casting the metal alloy into a blank, rod, tube, etc., 2) melting the metal alloy and/or metals that form the metal alloy, forming a metal strip, and then rolling and welding the strip into a blank, rod, tube, etc., 3) consolidating the metal powder of the metal alloy and/or metal powder of metals that form the metal alloy into a blank, rod, tube, etc., or 4) 3-D printing the metal powder of the metal alloy and/or metal powder of metals that form the metal alloy into a blank, rod, tube, etc.

In accordance with another and/or alternative aspect of the present disclosure, when the metal powder is consolidated to form the metal alloy into a blank, rod, tube, etc., the metal powder is pressed together to form a solid solution of the metal alloy into a near net component of the polyaxial pedicle screw arrangement. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.), and/or under a vacuum; however, this is not required.

In accordance with another and/or alternative aspect of the present disclosure, when metal powder is used to 3D print one or more portions of metal portion of the polyaxial pedicle screw arrangement, the average particle size of the metal powder is optionally 2-62 microns, and more particularly about 5-49.9 microns, the average density of the metal powders is greater than 5 g/cm³, and the metal powder is generally spherical-shaped, and the Hall flow (s/50 g) is less than 30 seconds (e.g., 2-29.99 seconds and all values and ranges therebetween). In another non-limiting embodiment of the disclosure, the average tensile elongation of the metal alloy used to partially or fully form the polyaxial pedicle screw arrangement is optionally at least about 25% (e.g., 25%-50% average tensile elongation and all values and ranges therebetween).

In accordance with another and/or alternative non-limiting aspect of the present disclosure, one or more portions of metal portion of the polyaxial pedicle screw arrangement can be partially (e.g., 1% to 99.99% and all values and ranges therebetween) or fully be coated with an enhancement layer to improve one or more properties of the polyaxial pedicle screw arrangement (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement layer forms a surface that is less of an irritant to cell about the coated surface after the polyaxial pedicle screw arrangement is implanted, reduces the rate to which cells grown on coated surface after the polyaxial pedicle screw arrangement is implanted, reduce rate to which movable components in the polyaxial pedicle screw arrangement fail to properly operate after orthopedic device is implanted, etc.).

In accordance with another and/or alternative non-limiting aspect of the present disclosure, non-limiting enhancement layers that can be applied to a portion or all of the outer surface of the polyaxial pedicle screw arrangement includes chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium oxynitride (ZrNxOy) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), and combinations of such coatings. In one non-limiting embodiment, the one or more enhancement layers are optionally applied to a portion or all of the outer surface of the polyaxial pedicle screw arrangement by a vacuum process using an energy source to vaporize material and deposit a thin layer of enhancement layer material. Such vacuum coating process, when used, can include a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In one non-limiting embodiment, the coating process is one or more of a PVD, CVD, ALD and PE-CVD, and wherein the coating process occurs at a temperature of 200-400° C. (and all values and ranges therebetween) for at least 10 minutes (e.g., 10-400 minutes and all values and ranges therebetween). In another non-limiting embodiment, the coating process is one or more of a PVD, CVD, ALD and PE-CVD, and wherein the coating process occurs at a temperature of 220-300° C. for 60-120 minutes. In another non-limiting embodiment, when the materials of the one or more enhancement layers are to be applied to the outer surface of the polyaxial pedicle screw arrangement that is partially or fully formed of a metal alloy, the materials of the one or more enhancement layers can optionally be combine with one or more metals in the metal alloy, and/or combined with nitrogen, oxygen, carbon, or other elements that are in the metal alloy and/or present in the atmosphere about the metal alloy to a form an enhancement layer on the outer surface of the metal alloy. In another non-limiting embodiment, when the materials of the one or more enhancement layers are to be applied to the outer surface of the polyaxial pedicle screw arrangement that is partially or fully formed of a metal alloy, the materials of the one or more enhancement layers can optionally be used to form various coating colors on the outer surface of the metal alloy (e.g., gold, copper, brass, black, rose gold, chrome, blue, silver, yellow, green, etc.). In another non-limiting embodiment, the thickness of the enhancement layer is greater than 1 nanometer (e.g., 2 nanometers to 100 microns and all values and ranges therebetween), and typically 0.1-25 microns, and more typically 0.2-10 microns. In another non-limiting embodiment, the hardness of the enhancement layer can be at least 5 GPa (ASTM C1327-15 or ASTM C1624-05), typically 5-50 GPa (and all values and ranges therebetween), more typically 10⁻²⁵ GPa, and still more typically 14-24 GPa. In another non-limiting embodiment, the coefficient of friction (COF) of the enhancement layer can be 0.04-0.2 (and all values and ranges therebetween), and typically 0.6-0.15. In another non-limiting embodiment, the wear rate of the enhancement layer can be 0.5×10⁻⁷ mm³/N−m to 3×10⁻⁷ mm³/N−m (and all values and ranges therebetween), and typically 1.2×10⁻⁷ mm³/N−m to 2×10⁻⁷ mm³/N−m. In another non-limiting embodiment, silicon-based precursors (e.g., trimethysilane, tetramethylsilane, hexachlorodisilane, silane, dichlorosilane, trichlorosilane, silicon tetrachloride, tris(dimethylamino) silane, bis(tert-butylamino)silane, trisilylamine, allyltrimethoxysilane, (3-aminopropyl)triethoxysilane, butyltrichlorosilane, n-sec-butyl(trimethylsilyl)amine, chloropentamethyldisilane, 1,2-dichlorotetramethyldisilane, [3-(diethylamino)propyl]trimethoxysilane, 1,3-diethyl-1,1,3,3-tetramethyldisilazane, dimethoxydimethylsilane, dodecamethylcyclohexasilane, hexamethyldisilane, isobutyl(trimethoxy)silane, methyltrichlorosilane, 2,4,6,8,10-pentamethylcyclopentasiloxane, pentamethyldisilane, n-propyltriethoxysilane, silicon tetrabromide, silicon tetrabromide, etc.) can optionally be used to facilitate in the application of the enhancement layer to one or more portions or all of the polyaxial pedicle screw arrangement. In one non-limiting embodiment, the enhancement layer includes no more than 0.1 wt. % nickel, no more than 0.1 wt. % chromium, and/or no more than 0.1 wt. % cobalt. In another non-limiting embodiment, the outer surface of the metal portion of the polyaxial pedicle screw arrangement includes no more than 0.1 wt. % nickel, no more than 0.1 wt. % chromium, and/or no more than 0.1 wt. % cobalt. The adhesion layer, when used, includes no more than 0.1 wt. % nickel, no more than 0.1 wt. % chromium, and/or no more than 0.1 wt. % cobalt.

In accordance with another and/or alternative non-limiting aspect of the present disclosure, one or more portions and/or components of the polyaxial pedicle screw arrangement can be partially or fully coated with an enhancement layer composition that includes a chromium nitride (CrN) coating. A portion or all of the polyaxial pedicle screw arrangement can be partially or fully coated with the chromium nitride (CrN) coating. The enhancement layer can be used to improve hardness, improve toughness, reduced friction, resistant impact wear, improve resistance to corrosion and oxidation, and/or form a reduced stick surface when in contact with many different materials. In accordance with one non-limiting embodiment, the chromium nitride (CrN) coating generally includes 40-85 wt. % Cr (and all values and ranges therebetween), 15-60 wt. % N (and all values and ranges therebetween), 0-10 wt. % Re (and all values and ranges therebetween), 0-10 wt. % Si (and all values and ranges therebetween), 0-2 wt. % O (and all values and ranges therebetween), and 0-2 wt. % C (and all values and ranges therebetween). In one non-limiting coating process, one or more portions and/or components of the polyaxial pedicle screw arrangement are initially coated with Cr metal. The Cr metal coating can be applied by PVD, CVD, ALD and PE-CVD in an inert environment. The coating thickness of Cr metal is 0.5-15 microns. Thereafter, the Cr metal coating is exposed to nitrogen gas and/or a nitrogen containing gas compound to cause the nitrogen to react with the Cr metal coating to form a layer of CrN on the outer surface of the Cr metal coating and/or the outer surface of one or more components of the polyaxial pedicle screw arrangement. Particles of Cr metal can optionally be mixed with nitrogen gas and/or a nitrogen containing gas compound to facilitate in the formation of the CrN coating. When Cr metal particles are used, the initial Cr coating layer on the outer surface of one or more components of the polyaxial pedicle screw arrangement can optionally be eliminated. In another non-limiting embodiment, the enhancement layer composition generally includes 65-80 wt. % Cr, 15-30 wt. % N, 0-8 wt. % Re, 0-1 wt. % Si, 0-1 wt. % O, and 0-1 wt. % C.

In accordance with another and/or alternative non-limiting aspect of the present disclosure, one or more portions and/or components of the polyaxial pedicle screw arrangement can be partially or fully coated with an enhancement layer composition that includes a diamond-Like Carbon (DLC) coating. A portion or all of the polyaxial pedicle screw arrangement can be partially or fully coated with the diamond-Like Carbon (DLC) coating. The enhancement layer can be used to improve hardness, improve toughness, reduced friction, resistant impact wear, improve resistance to corrosion and oxidation, improve biocompatibility, and/or form a reduced stick surface when in contact with many different materials. In one non-limiting embodiment, the diamond-Like Carbon (DLC) coating generally includes 60-99.99 wt. % C (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % O (and all values and ranges therebetween). The carbon coating can be applied by PVD, CVD, ALD and PE-CVD in an inert environment. The carbon layer can be applied by using methane and/or acetylene gas; however, other or additional carbon sources can be used. The coating thickness of the carbon is 0.5-15 microns. In another non-limiting embodiment, one or more portions and/or components of the polyaxial pedicle screw arrangement are coated with the enhancement layer composition that generally includes 90-99.99 wt. % C, 0-1 wt. % N, 0-8 wt. % Re, 0-1 wt. % Si, and 0-1 wt. % O.

In accordance with another and/or alternative non-limiting aspect of the present disclosure, one or more portions and/or components of the polyaxial pedicle screw arrangement can be partially or fully coated with an enhancement layer composition that includes a titanium nitride (TiN) coating. A portion or all of the outer surface of the polyaxial pedicle screw arrangement can include the titanium nitride (TiN) coating. The enhancement layer can be used to improve hardness, improve toughness, improve resistance to corrosion and oxidation, reduced friction, and/or form a reduced stick surface when in contact with many different materials. In one non-limiting embodiment, one or more portions and/or components of the polyaxial pedicle screw arrangement are optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating, when applied, is exposed to nitrogen gas and/or a nitrogen containing gas compound and optionally titanium particles to cause the nitrogen to react with the Ti metal coating and/or titanium metal particles to form a layer of TiN on the outer surface of the Ti metal coating and/or the outer surface of the one or more portions or components of the polyaxial pedicle screw arrangement. If a titanium layer is not preapplied, the TiN coating can be formed by exposing the outer surface of one or more portions or components of the polyaxial pedicle screw arrangement to titanium particles and nitrogen gas and/or a nitrogen containing gas compound. The coating thickness of the TiN coating is generally at least 0.1 microns (e.g., 0.1-15 microns and all values and ranges therebetween), and typically 0.2-2 microns.

In accordance with another and/or alternative non-limiting aspect of the present disclosure, one or more portions and/or components of the polyaxial pedicle screw arrangement can be partially or fully coated with an enhancement layer composition that includes a titanium oxynitride or titanium nitride oxide (TiNOx) coating. A portion or all of the outer surface of the one or more portions or components of the polyaxial pedicle screw arrangement can include the titanium oxynitride or titanium nitride oxide (TiNOx) coating. The enhancement layer can be used to improve hardness, improve toughness, improve resistance to corrosion and oxidation, reduced friction, and/or form a reduced stick surface when in contact with many different materials, and/or promote nitric oxide formation on the surface of the coating. In one non-limiting embodiment, all or a portion of the outer surface of the polyaxial pedicle screw arrangement is optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating is exposed to titanium particles and a nitrogen and oxygen mixture that can include nitrogen gas, oxygen gas, a nitrogen containing gas compound and/or an oxygen containing gas compound to cause the nitrogen and oxygen to react with the Ti metal coating, if such coating is used, and/or with the Ti metal particles to form a layer of TiNOx on the outer surface of the Ti metal coating and/or the outer surface of one or more portions or components of the polyaxial pedicle screw arrangement. The ratio of the N to the O can be varied to control the amount of O in the TiNOx coating. If a titanium layer is not preapplied, the TiNOx coating can be formed by exposing one or more portions or components of the polyaxial pedicle screw arrangement to titanium particles and a nitrogen and oxygen source such as nitrogen gas, oxygen gas, a nitrogen containing gas compound and/or an oxygen containing gas compound. The ratio of N to O when forming the TiNOx coating is generally 1:10 to 10:1 (and all values and ranges therebetween). The coating thickness of the TiNOx coating is generally at least 0.1 microns (e.g., 0.1-15 microns and all values and ranges therebetween), and typically 0.2-2 microns. In another non-limiting embodiment, a TiNOx coating is applied to a portion or all of the outer surface of the one or more portions or components of the polyaxial pedicle screw arrangement, and the TiNOx coating is formed by a) exposing the outer surface of a portion of all of the one or more portions or components of the polyaxial pedicle screw arrangement to Ti particles (PVD, CVD, ALD and PE-CVD process) and/or a Ti containing solution to form a Ti layer on a portion of all of the one or more portions or components of the polyaxial pedicle screw arrangement, and wherein the thickness of the Ti coating is 0.05-5 microns, and b) exposing the Ti coating to a nitrogen and oxygen source such as nitrogen gas, oxygen gas, a nitrogen containing gas compound and/or an oxygen containing gas compound to form a TiNOx coating, and wherein ratio of N to O when forming the TiNOx coating is generally 1:10 to 10:1, and wherein the coating thickness of the TiNOx coating is 0.2-5 microns. In another non-limiting embodiment, a TiNOx coating is applied to a portion or all of the outer surface of the one or more portions or components of the polyaxial pedicle screw arrangement, and the TiNOx coating is formed by exposing a portion or all of the outer surface of the polyaxial pedicle screw arrangement to Ti particles and a nitrogen and oxygen source such as nitrogen gas, oxygen gas, a nitrogen containing gas compound and/or an oxygen containing gas compound to form a TiNOx coating, and wherein ratio of N to O when forming the TiNOx coating is generally 1:10 to 10:1, and wherein the coating thickness of the TiNOx coating is 0.2-5 microns. In another non-limiting embodiment, the enhancement layer composition generally includes 20-85 wt. % Ti (and all values and ranges therebetween), 0.5-35 wt. % N (and all values and ranges therebetween), 0-10 wt. % Re (and all values and ranges therebetween), and 0.5-35 wt. % O (and all values and ranges therebetween). In another non-limiting embodiment, a coating of TiNOx was formed on one or more portions or components of the polyaxial pedicle screw arrangement by reactive physical vapor deposition in a vacuum chamber. Depending on the oxygen-nitrogen ratio during vapor deposition, a coating deposit of TiNOx with defined composition and resistivity can be coated on the outer surface of the one or more portions or components of the polyaxial pedicle screw arrangement.

In accordance with another and/or alternative non-limiting aspect of the present disclosure, one or more portions and/or components of the polyaxial pedicle screw arrangement can be partially or fully coated with an enhancement layer composition that includes a zirconium nitride (ZrN) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the one or more portions or components of the polyaxial pedicle screw arrangement is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound to cause the nitrogen to react with the Zn metal coating to form a layer of ZrN on the outer surface of the Zr metal coating and/or the outer surface of one or more portions or components of the polyaxial pedicle screw arrangement. Particles of Zr metal can optionally be mixed with nitrogen gas and/or a nitrogen containing gas compound to facilitate in the formation of the ZrN coating. When Zr metal particles are used, the initial Zr coating layer on the outer surface of one or more portions or components of the polyaxial pedicle screw arrangement can optionally be eliminated. The ZrN coating has been found to produce a gold-colored enhancement layer color. In another non-limiting embodiment, the enhancement layer composition generally includes 35-90 wt. % Zr (and all values and ranges therebetween), 5-25 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another non-limiting embodiment, the enhancement layer composition generally includes 80-90 wt. % Zr, 10⁻²⁰ wt. % N, 0-8 wt. % Re, 0-1 wt. % Si, 0-1 wt. % O, and 0-1 wt. % C.

In accordance with another and/or alternative non-limiting aspect of the present disclosure, one or more portions and/or components of the polyaxial pedicle screw arrangement includes a zirconium oxide (ZrO₂) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the one or more portions or components of the polyaxial pedicle screw arrangement is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to oxygen gas and/or oxygen containing gas compound to cause the oxygen to react with the Zn metal coating to form a layer of zirconium oxide (ZrO₂) on the outer surface of the Zr metal coating and/or the outer surface of the one or more portions or components of the polyaxial pedicle screw arrangement. Particles of Zr metal can optionally be mixed with oxygen gas and/or an oxygen containing gas compound to facilitate in the formation of the ZrO₂ coating. When Zr metal particles are used, the initial Zr coating layer on the outer surface of one or more portions or components of the polyaxial pedicle screw arrangement can optionally be eliminated. The zirconium oxide (ZrO₂) coating has been found to produce a blue colored enhancement layer color. In another non-limiting embodiment, the enhancement layer composition generally includes 35-90 wt. % Zr (and all values and ranges therebetween), 10⁻³⁵ wt. % O (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % C (and all values and ranges therebetween). In another non-limiting embodiment, the enhancement layer composition generally includes 70-80 wt. % Zr, 20-30 wt. %, 0-1 wt. % N, 0-8 wt. % Re, 0-1 wt. % Si, and 0-1 wt. % C.

In accordance with another and/or alternative non-limiting aspect of the present disclosure, one or more portions and/or components of the polyaxial pedicle screw arrangement includes both a zirconium oxide (ZrO₂) coating and a zirconium nitride coating (ZrN). The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of one or more portions or components of the polyaxial pedicle screw arrangement is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both oxygen gas and/or oxygen containing gas compound and also to nitrogen gas and/or nitrogen containing gas compound, b) nitrogen gas and/or nitrogen containing gas compound and then to oxygen gas and/or oxygen containing gas compound, or c) oxygen gas and/or oxygen gas containing compound and then to nitrogen gas and/or nitrogen gas containing compound. The coating composition of the zirconium oxide (ZrO₂) coating and the zirconium nitride coating (ZrN) are similar or the same as discussed above. As discussed above, Particles of Zr metal can optionally be mixed with oxygen gas and/or an oxygen containing gas compound to facilitate in the formation of the ZrO₂ coating and the nitrogen gas and/or nitrogen gas containing compound to facilitate in the formation of the ZrN coating. When Zr metal particles are used, the initial Zr coating layer on the outer surface of one or more portions or components of the polyaxial pedicle screw arrangement can optionally be eliminated.

In accordance with another and/or alternative non-limiting aspect of the present disclosure, one or more portions and/or components of the polyaxial pedicle screw arrangement includes a zirconium oxycarbide (ZrOC) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of one or more portions or components or the polyaxial pedicle screw arrangement is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both to oxygen gas and/or an oxygen containing gas compound and to carbon and/or a carbon containing gas compound (e.g., methane and/or acetylene gas), b) carbon and/or a carbon containing gas compound and then to oxygen gas and/or an oxygen containing gas compound, or c) oxygen gas and/or oxygen containing gas compound and then to carbon and/or carbon containing gas compound. Particles of Zr metal can optionally be mixed with oxygen gas and/or an oxygen containing gas compound and the carbon and/or carbon containing gas compound to facilitate in the formation of the zirconium oxycarbide (ZrOC) coating. When Zr metal particles are used, the initial Zr coating layer on the outer surface of one or more portions or components of the polyaxial pedicle screw arrangement can optionally be eliminated. In another non-limiting embodiment, the enhancement layer composition generally includes 40-95 wt. % Zr (and all values and ranges therebetween), 5-25 wt. % O (and all values and ranges therebetween), and 10⁻⁴⁰ wt. % C (and all values and ranges therebetween), 0-2 wt. % N (and all values and ranges therebetween), 0-10 wt. % Re (and all values and ranges therebetween), and 0-20 wt. % Si (and all values and ranges therebetween). In another non-limiting embodiment, the enhancement layer composition generally includes 40-65 wt. % Zr, 5-25 wt. % O, and 25-40 wt. % C, 0-1 wt. % N, 0-8 wt. % Re, and 0-1 wt. % Si.

In accordance with another and/or alternative non-limiting aspect of the present disclosure, one or more portions and/or components of the polyaxial pedicle screw arrangement includes a zirconium oxynitride (ZrNxOy) [e.g., cubic ZrN:O, cubic ZrO₂:N, tetragonal ZrO₂:N, and monoclinic ZrO₂:N phase coatings]. A portion or all of the outer surface of the one or more portions or components of the polyaxial pedicle screw arrangement can include the zirconium oxynitride (ZrNxOy). The enhancement layer can be used to improve hardness, improve toughness, improve resistance to corrosion and oxidation, reduced friction, form a reduced stick surface when in contact with many different materials, and/or promote nitric oxide formation on the surface of the coating. In one non-limiting embodiment, all or a portion of the outer surface of the one or more portions or components of the polyaxial pedicle screw arrangement are optionally initially coated with Zr metal. The Zr metal coating, when applied, can be applied by PVD, CVD, ALD and PE-CVD in an inert environment. The coating thickness of Zr metal is 0.05-15 microns (and all values and ranges therebetween). As can be appreciated, the initial Zr coating is optional. Thereafter, the Zr metal coating is exposed to zirconium particles and a nitrogen and oxygen mixture that can include nitrogen gas, oxygen gas, a nitrogen containing gas compound and/or an oxygen containing gas compound to cause the nitrogen and oxygen to react with the Zr metal coating, if such coating is used, and/or with the Zr metal particles to form a layer of ZrNxOy on the outer surface of the Zr metal coating and/or the outer surface of the one or more portions or components of the polyaxial pedicle screw arrangement. The ratio of the N to the O can be varied to control the amount of O and N in the ZrNxOy coating. If a zirconium layer is not preapplied, the ZrNxOy coating can be formed by exposing the outer surface of one or more portions or components of the polyaxial pedicle screw arrangement to zirconium particles and a nitrogen and oxygen source such as nitrogen gas, oxygen gas, a nitrogen containing gas compound and/or an oxygen containing gas compound. The ratio of N to O when forming the ZrNxOy coating is generally 1:10 to 10:1 (and all values and ranges therebetween). The coating thickness of the ZrNxOy coating is generally at least 0.1 microns (e.g., 0.1-15 microns and all values and ranges therebetween), and typically 0.2-2 microns. In another non-limiting embodiment, a ZrNxOy coating is applied to a portion or all of the outer surface of the one or more portions or components of the polyaxial pedicle screw arrangement, and the ZrNxOy coating is formed by a) exposing the outer surface of a portion of all of the one or more portions or components of the polyaxial pedicle screw arrangement to Zr particles (PVD, CVD, ALD and PE-CVD process) and/or a Zr containing solution to form a Zr layer on a portion of all of the one or more portions or components of the polyaxial pedicle screw arrangement, and wherein the thickness of the Zr coating is 0.05-5 microns, and b) exposing the Zr coating to a nitrogen and oxygen source such as nitrogen gas, oxygen gas, a nitrogen containing gas compound and/or an oxygen containing gas compound to form a ZrNxOy coating, and wherein ratio of N to O when forming the ZrNxOy coating is generally 1:10 to 10:1, and wherein the coating thickness of the ZrNxOy coating is 0.2-5 microns. In another non-limiting embodiment, a ZrNxOy coating is applied to a portion or all of the outer surface of the one or more portions or components of the polyaxial pedicle screw arrangement, and the ZrNxOy coating is formed by exposing a portion or all of the outer surface of the one or more portions or components of the polyaxial pedicle screw arrangement to Zr particles and a nitrogen and oxygen source such as nitrogen gas, oxygen gas, a nitrogen containing gas compound and/or an oxygen containing gas compound to form a ZrNxOy coating, and wherein ratio of N to O when forming the ZrNxOy coating is generally 1:10 to 10:1, and wherein the coating thickness of the ZrNxOy coating is 0.2-5 microns. In another non-limiting embodiment, the enhancement layer composition generally includes 20-85 wt. % Zr (and all values and ranges therebetween), 0.5-35 wt. % N (and all values and ranges therebetween), and 0.5-35 wt. % O (and all values and ranges therebetween). In another non-limiting embodiment, a coating of ZrNxOy was formed on one or more portions or components of the polyaxial pedicle screw arrangement by reactive physical vapor deposition in a vacuum chamber. Depending on the oxygen-nitrogen ratio during vapor deposition, a coating deposit of ZrNxOy with defined composition and resistivity can be coated on the outer surface of the one or more portions or components of the polyaxial pedicle screw arrangement.

In accordance with another and/or alternative non-limiting aspect of the present disclosure, one or more portions and/or components of the polyaxial pedicle screw arrangement includes a zirconium-nitrogen-carbon (ZrNC) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the one or more portions or components of the polyaxial pedicle screw arrangement is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound and then to carbon and/or a carbon containing gas compound (e.g., methane and/or acetylene gas). The color of the ZrNC will vary depending on the amount of C and N in the coating. Particles of Zr metal can optionally be mixed with nitrogen gas and/or a nitrogen containing gas compound and the carbon and/or a carbon containing gas compound to facilitate in the formation of the ZrNC coating. When Zr metal particles are used, the initial Zr coating layer on the outer surface of one or more portions or components of the polyaxial pedicle screw arrangement can optionally be eliminated. In one non-limiting embodiment, the enhancement layer composition generally includes 40-95 wt. % Zr (and all values and ranges therebetween), 5-40 wt. % N (and all values and ranges therebetween), and 5-40 wt. % C (and all values and ranges therebetween), 0-2 wt. % O (and all values and ranges therebetween), 0-10 wt. % Re (and all values and ranges therebetween), and 0-20 wt. % Si (and all values and ranges therebetween). In another non-limiting embodiment, the enhancement layer composition generally includes 40-80 wt. % Zr, 5-25 wt. % N, and 5-25 wt. % C, 0-1 wt. % O, 0-8 wt. % Re, and 0-1 wt. % Si.

In accordance with another and/or alternative non-limiting aspect of the present disclosure, a portion or all of the polyaxial pedicle screw arrangement is formed of a metal alloy that includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, 1) refractory metal alloy, or m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 awt. %, and wherein a portion or all of the outer surface of the metal alloy is coated with an enhancement layer (e.g., chromium nitride (CrN), diamond-like carbon (DLC), titanium nitride (TiN), titanium nitride oxide (TiNOx), zirconium nitride (ZrN), zirconium oxide (ZrO₂), zirconium-nitrogen-carbon (ZrNC), zirconium OxyCarbide (ZrOC), zirconium oxynitride (ZrNxOy) [e.g., cubic ZrN:O, cubic ZrO₂:N, tetragonal ZrO₂:N, and monoclinic ZrO₂:N phase coatings]), and wherein the outer surface of the metal alloy optionally includes an adhesion layer, which adhesion layer is optionally a metallic layer that includes titanium or zirconium.

In accordance with another and/or alternative non-limiting aspect of the present disclosure, a portion or all of the polyaxial pedicle screw arrangement is formed of a metal alloy that includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, or m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 awt. %, and wherein the metal alloy is coated with a metal oxynitride layer (e.g., titanium nitride oxide and/or (TiNOx), zirconium oxynitride (ZrNxOy), etc.), which metal oxynitride layer can optionally be used to promotes and/or facilitates in a) formation or generation of nitric oxide (NO), b) stimulation of endothelial cells, c) a modulation of endothelial cells, d) reduce neointimal hyperplasia, e) reduce tissue proliferation, f) reduce platelet activation, g) reduce thrombosis, h) reduce restenosis, i) promote endothelial cell angiogenesis, and/or j) improved healing on and/or about the polyaxial pedicle screw arrangement, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the polyaxial pedicle screw arrangement is formed of a titanium-nickel alloy or a titanium-nickel alloy that includes at least 5 awt. % rhenium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion the polyaxial pedicle screw arrangement are optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the medical is formed of a stainless-steel alloy or a stainless-steel alloy that includes at least 5 awt. % rhenium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the polyaxial pedicle screw arrangement is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the polyaxial pedicle screw arrangement is formed of a cobalt-chromium alloy or a cobalt-chromium alloy that includes at least 5 awt. % rhenium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of components of the polyaxial pedicle screw arrangement is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the polyaxial pedicle screw arrangement is formed of a TiAlV alloy or a TiAlV alloy that includes at least 5 awt. % rhenium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the polyaxial pedicle screw arrangement is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the polyaxial pedicle screw arrangement is formed of a refractory metal alloy or a refractory metal alloy that includes at least 5 awt. % rhenium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the polyaxial pedicle screw arrangement is optionally coated with a metal oxynitride layer, and wherein the outer surface of the metal alloy optionally includes an adhesion layer, which adhesion layer is optionally a metallic layer that includes titanium or zirconium, and which metal oxynitride layer is optionally partially or fully coated on the outer surface of the adhesion layer.

In accordance with another and/or alternative non-limiting aspect of the present disclosure, all or a portion of the polyaxial pedicle screw arrangement is formed of a metal alloy that includes at least 5 awt. % rhenium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of the polyaxial pedicle screw arrangement optionally coated with a metal oxynitride layer, and wherein the outer surface of the polyaxial pedicle screw arrangement that includes the metal oxynitride layer optionally includes an adhesion layer, which adhesion layer is optionally a metallic layer that includes titanium or zirconium, and which metal oxynitride layer is optionally partially or fully coated on the outer surface of the adhesion layer.

One non-limiting object of the present disclosure is the provision of a polyaxial pedicle screw arrangement that can be temporarily locked in position, thereby temporarily to thereby convert the polyaxial pedicle screw arrangement into a monoaxial-behaving pedicle screw arrangement.

Another and/or alternative non-limiting object of the present disclosure is the provision of a polyaxial pedicle screw arrangement that includes a tulip, an upper pressure cap, a lower pressure cap/flexible collet and a pedicle screw, and wherein the polyaxial pedicle screw arrangement that can be temporarily locked in in position, thereby temporarily to thereby convert the polyaxial pedicle screw arrangement in to a monoaxial-behaving pedicle screw arrangement.

Another and/or alternative non-limiting object of the present disclosure is the provision of a polyaxial pedicle screw arrangement that can be temporarily locked in position during a surgical procedure; the polyaxial pedicle screw arrangement comprising a tulip, an upper pressure cap, a lower pressure cap/flexible collet and a pedicle screw; the upper pressure cap and/or the lower pressure cap/flexible collet are configured to move within the tulip when a force is applied to the upper pressure cap and/or the lower pressure cap/flexible collet, and wherein the movement of the upper pressure cap and/or the lower pressure cap/flexible collet in at least one direction within the tulip is configured to inhibit or prevent movement of the pedicle screw relative to the upper pressure cap and/or the lower pressure cap/flexible collet.

Another and/or alternative non-limiting object of the present disclosure is the provision of a polyaxial pedicle screw arrangement wherein the upper pressure cap and/or the lower pressure cap/flexible collet are configured to move upwardly and downwardly along a longitudinal axis of the tulip, and wherein downward movement of the upper pressure cap and/or the lower pressure cap/flexible collet within the tulip is configured to inhibit or prevent movement of the pedicle screw relative to the upper pressure cap and/or the lower pressure cap/flexible collet.

Another and/or alternative non-limiting object of the present disclosure is the provision of a polyaxial pedicle screw arrangement wherein the upper pressure cap is positioned above the lower pressure cap/flexible cap; the upper pressure cap is configured to engage the lower pressure cap/flexible cap.

Another and/or alternative non-limiting object of the present disclosure is the provision of a polyaxial pedicle screw arrangement wherein the pedicle screw includes a head portion and a body portion; the head portion has a maximum cross-sectional area that is greater than a maximum cross-sectional area of the body portion.

Another and/or alternative non-limiting object of the present disclosure is the provision of a polyaxial pedicle screw arrangement wherein the tulip includes body that has a top portion and a bottom portion; the top portion including a top cavity; the bottom portion including a bottom cavity; the top portion including two top side openings configured to receive a portion of an orthopedic rod or other type of medical device; the two top side openings forming first and second upwardly extending arms; the top cavity including a top securing surface configured to receive a connector to entrap the orthopedic rod or other type of medical device in the two top side openings; the bottom cavity configured to receive at least a portion of the lower pressure cap/flexible collet and/or the pedicle screw.

Another and/or alternative non-limiting object of the present disclosure is the provision of a polyaxial pedicle screw arrangement wherein the top securing surface includes a threaded surface.

Another and/or alternative non-limiting object of the present disclosure is the provision of a polyaxial pedicle screw arrangement wherein the body of the tulip includes a mid-opening that is positioned along a longitudinal axis of the body; the opening forming a passageway between the top and bottom cavities.

Another and/or alternative non-limiting object of the present disclosure is the provision of a polyaxial pedicle screw arrangement wherein a maximum cross-sectional area of the mid-opening is less than a maximum cross-sectional area of the head portion of the pedicle screw.

Another and/or alternative non-limiting object of the present disclosure is the provision of a polyaxial pedicle screw arrangement wherein the body of the tulip includes one or more side slots that are configured to receive a portion of the upper pressure cap; the one or more side slots are configured to limit movement of the upper pressure cap along the longitudinal axis of the tulip when the polyaxial pedicle screw arrangement is fully assembled.

Another and/or alternative non-limiting object of the present disclosure is the provision of a polyaxial pedicle screw arrangement wherein the lower pressure cap/flexible cap includes a body that has a body central axial cavity; a lower portion of the body central cavity has a cross-sectional area such that the head portion of the pedicle screw can at least partially pass into the body central axial cavity.

Another and/or alternative non-limiting object of the present disclosure is the provision of a polyaxial pedicle screw arrangement wherein the upper pressure cap includes one or more side slots that are configured to receive a portion of the lower pressure cap/flexible collet; the one or more side slots of the upper pressure cap are configured to limit rotational movement of the lower pressure cap/flexible collet about a central longitudinal axis of the tulip when the pedicle screw arrangement is fully assembled.

Another and/or alternative non-limiting object of the present disclosure is the provision of a polyaxial pedicle screw arrangement wherein the lower pressure cap/flexible collet includes one or more position flanges that extend upwardly from the body of the lower pressure cap/flexible cap, and wherein the one or more position flanges are configured to engage one or more side slots of the upper pressure cap.

Another and/or alternative non-limiting object of the present disclosure is the provision of a polyaxial pedicle screw arrangement wherein the body of the lower pressure cap/flexible collet includes one or more slots that extend from a base of the body to a point that is spaced from an upper edge of the body; the one or more slots are configured to enable a lower portion of the body to be compressed and to reduce in cross-sectional area when a downward force is applied to the lower pressure cap/flexible collet.

Another and/or alternative non-limiting object of the present disclosure is the provision of a medical instrument for use with the polyaxial pedicle screw wherein the medical instrument includes one or more engagement legs that are configured to engage a portion of the upper pressure cap and/or the lower pressure cap/flexible collet to cause movement of the upper pressure cap and/or the lower pressure cap/flexible collet.

Another and/or alternative non-limiting object of the present disclosure is the provision of a medical instrument for use with the polyaxial pedicle screw wherein the one or more engagement legs are configured to engage one or more engagement areas located on a top surface of the upper pressure cap; each of the one or more engagement areas is configured to receive a portion of one of the engagement legs.

Another and/or alternative non-limiting object of the present disclosure is the provision of a medical instrument for use with the polyaxial pedicle screw wherein the medical instrument includes an outer body and an inner body; the inner body is configured to be rotatably positioned in the outer body and rotatable about a central longitudinal axis of the medical instrument; a top portion of the inner body extends upwardly from a top portion of the outer body; a top portion of the inner body includes one or more engagement members that are configured to be engaged by a medical tool to facilitate in rotation of the inner body relative to the outer body; a bottom portion of the outer body is configured to releasably engage an outer surface of the tulip; the outer body includes one or more side openings used to enable a user to a) view movement of one or more portions of the inner body within the outer body, b) view a distance of longitudinal movement of the inner body within the outer body, and/or c) view a positioning of the engagement legs relative to the engagement areas on the upper pressure cap.

Another and/or alternative non-limiting object of the present disclosure is the provision of a polyaxial pedicle screw arrangement that can be temporarily locked in position during a surgical procedure; the polyaxial pedicle screw arrangement comprising a tulip, an upper pressure cap, and a lower pressure cap/flexible collet; the upper pressure cap and/or the lower pressure cap/flexible collet are configured to move within the tulip when a force is applied to the upper pressure cap and/or the lower pressure cap/flexible collet, and wherein the movement of the upper pressure cap and/or the lower pressure cap/flexible collet in at least one direction within the tulip is configured to inhibit or prevent movement of a pedicle screw relative to the upper pressure cap and/or the lower pressure cap/flexible collet.

Another and/or alternative non-limiting object of the present disclosure is the provision of a method for using a polyaxial pedicle screw arrangement; the method comprising: 1) providing the polyaxial pedicle screw arrangement; 2) providing a medical instrument for use with a polyaxial pedicle screw arrangement; the medical instrument includes an outer body and an inner body; the inner body is configured to be rotatably positioned in the outer body and rotatable about a central longitudinal axis of the medical instrument; a top portion of the inner body extends upwardly from a top portion of the outer body; a top portion of the inner body includes one or more engagement members that are configured to facilitate in rotation of the inner body relative to the outer body; a bottom portion of the outer body is configured to releasably engage an outer surface of the tulip; the outer body includes one or more side openings used to enable a user to a) view movement of one or more portions of the inner body within the outer body, b) view a distance of longitudinal movement of the inner body within the outer body, and/or c) view a positioning of the engagement legs relative to the engagement areas on the upper pressure cap; the medical instrument includes one or more engagement legs that are configured to engage a portion of the polyaxial pedicle screw arrangement; 3) engaging the polyaxial pedicle screw arrangement with the medical instrument to cause the one or more engagement members to engage the upper pressure cap and/or the lower pressure cap/flexible collet; and 4) causing the inner body to move downwardly relative to the outer body to cause the upper pressure cap and/or the lower pressure cap/flexible collet to move downwardly in the tulip and transform the polyaxial pedicle screw arrangement from a polyaxial moving arrangement to a monoaxial moving arrangement.

Another and/or alternative non-limiting object of the present disclosure is the provision of a polyaxial pedicle screw arrangement that is partially or fully formed of a metal alloy that includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, or m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 awt. %.

Another and/or alternative non-limiting object of the present disclosure is the provision of a polyaxial pedicle screw arrangement that is partially or fully coated with an enhancement material.

Another and/or alternative non-limiting object of the present disclosure is the provision of a polyaxial pedicle screw arrangement that is partially or fully coated with an enhancement layer (e.g., chromium nitride (CrN), diamond-like carbon (DLC), titanium nitride (TiN), titanium nitride oxide (TiNOx), zirconium nitride (ZrN), zirconium oxide (ZrO₂), zirconium-nitrogen-carbon (ZrNC), zirconium OxyCarbide (ZrOC), zirconium oxynitride (ZrNxOy) [e.g., cubic ZrN:O, cubic ZrO₂:N, tetragonal ZrO₂:N, and monoclinic ZrO₂:N phase coatings]), and wherein the outer surface of the metal alloy optionally includes an adhesion layer, which adhesion layer is optionally a metallic layer that includes titanium or zirconium.

Another and/or alternative non-limiting object of the present disclosure is the provision of a polyaxial pedicle screw arrangement that is partially or fully coated with an enhancement material that includes metal oxynitride.

Another and/or alternative non-limiting object of the present disclosure is the provision of a polyaxial pedicle screw arrangement that is partially or fully coated with an enhancement material that includes metal oxynitride, and wherein the metal oxynitrider includes titanium oxynitride and/or zirconium oxynitride.

Another and/or alternative non-limiting object of the present disclosure is the provision of a polyaxial pedicle screw arrangement that is partially or fully coated with an enhancement material that includes metal oxynitride, and wherein the metal oxynitrider layer has a thickness of at least 10 nanometers.

Another and/or alternative non-limiting object of the present disclosure is the provision of a polyaxial pedicle screw arrangement that is partially or fully coated with an enhancement material that includes metal oxynitride, and wherein the metal oxynitride has an oxygen to nitrogen atomic ratio of 1:10 to 10:1.

Another and/or alternative non-limiting object of the present disclosure is the provision of a polyaxial pedicle screw arrangement that includes an enhancement layer, and wherein the enhancement layer includes metal oxynitride and a metallic adhesion layer; and wherein the metal oxynitride layer is partially or fully coated on an outer surface of the metallic adhesion layer; and wherein the metallic adhesion layer is coated on an outer surface of one or more portions of the polyaxial pedicle screw arrangement.

Another and/or alternative non-limiting object of the present disclosure is the provision of a polyaxial pedicle screw arrangement that includes an enhancement layer, and wherein the enhancement layer includes metal oxynitride and a metallic adhesion layer; and wherein the metallic adhesion layer includes titanium metal or zirconium metal.

Another and/or alternative non-limiting object of the present disclosure is the provision of a polyaxial pedicle screw arrangement that includes an enhancement layer, and wherein the enhancement layer includes metal oxynitride and a metallic adhesion layer; and wherein the metallic adhesion layer has a thickness of at least 1 nanometer.

Another and/or alternative non-limiting object of the present disclosure is the provision of a polyaxial pedicle screw arrangement that includes an enhancement layer, and wherein the enhancement layer includes no more than 0.1 wt. % nickel, no more than 0.1 wt. % chromium, and/or no more than 0.1 wt. % cobalt.

Another and/or alternative non-limiting object of the present disclosure is the provision of a polyaxial pedicle screw arrangement that is at least partially formed of a metal material that includes no more than 0.1 wt. % nickel, no more than 0.1 wt. % chromium, and/or no more than 0.1 wt. % cobalt.

These and other objects and advantages will become apparent to those skilled in the art upon reading and following the description taken together with the accompanying drawings.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 illustrates a partial assembly of polyaxial pedicle screw arrangement 10 wherein the lower pressure cap/flexible cap 80 has been inserted onto the head portion 22 of the pedicle screw 20 and the upper pressure cap 60 is positioned on the top surface of the lower pressure cap/flexible cap 80.

FIG. 2 illustrates a fully assembled polyaxial pedicle screw arrangement 10.

FIG. 3 illustrates a fully assembled polyaxial pedicle screw arrangement 10, and the tulip 30 is drawn in a transparent form so as to illustrate the positioning of the upper pressure cap 60 and the lower pressure cap/flexible collet 80 in the tulip 30 when the polyaxial pedicle screw arrangement 10 is fully assembled.

FIG. 4 is an enlarge view of the upper portion of the fully assembled polyaxial pedicle screw arrangement 10 of FIG. 3.

FIGS. 5 and 6 are illustrations of the temporary locking of the polyaxial pedicle screw arrangement 10.

FIG. 7 illustrates the upper pressure cap 60 and the tulip 30 and how the upper pressure cap 60 is inserted into the tulip 30.

FIG. 8 illustrates the lower pressure cap/flexible cap 80 and the tulip 30 and how the lower pressure cap/flexible cap 80 is inserted into the tulip 30.

FIG. 9 illustrates the pedicle screw 20 and the tulip 30 and how the pedicle screw 20 is inserted into the tulip 30 when the lower pressure cap/flexible cap 80 has also been inserted into the tulip 30.

FIGS. 10 and 11 are cross-sectional views of an enlarge view of the upper portion of the fully assembled polyaxial pedicle screw arrangement 10 of FIG. 3.

FIG. 12 illustrates an orthopedic device OD in the form of a rod that is secured to tulip by a screw that is threaded to the tulip.

FIG. 13 illustrates a non-limiting medical instrument that can be used with the polyaxial pedicle screw arrangement.

FIG. 14 is an enlarged view of the bottom portion of the medical instrument that is in engagement with the tulip of polyaxial pedicle screw arrangement and wherein the bottom engagement legs of the inner body are spaced upwardly from engagement areas of the upper pressure cap.

FIG. 15 is an enlarged view of the bottom portion of the medical instrument that is in engagement with the tulip of polyaxial pedicle screw arrangement and wherein the bottom engagement legs of the inner body are engaged with the engagement areas of the upper pressure cap.

FIG. 16 is a similar view as in FIG. 15 except that the outer body of the medical instrument is not shown.

FIG. 17 is an illustration of inner body of the medical instrument.

### DESCRIPTION OF NON-LIMITING EMBODIMENTS

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

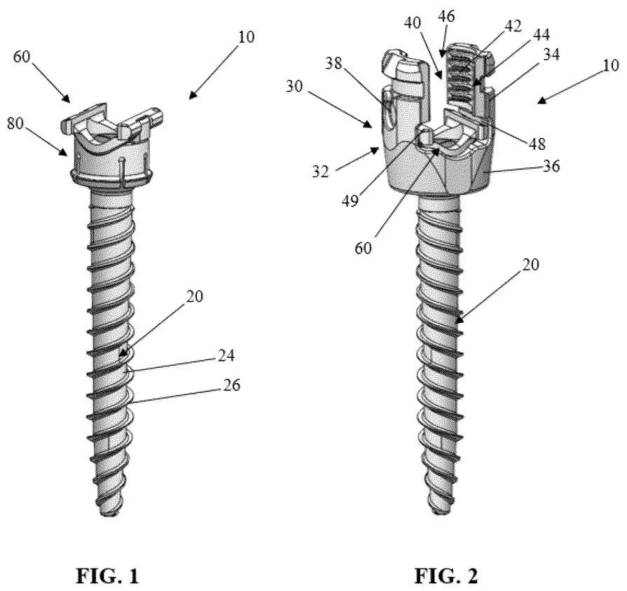

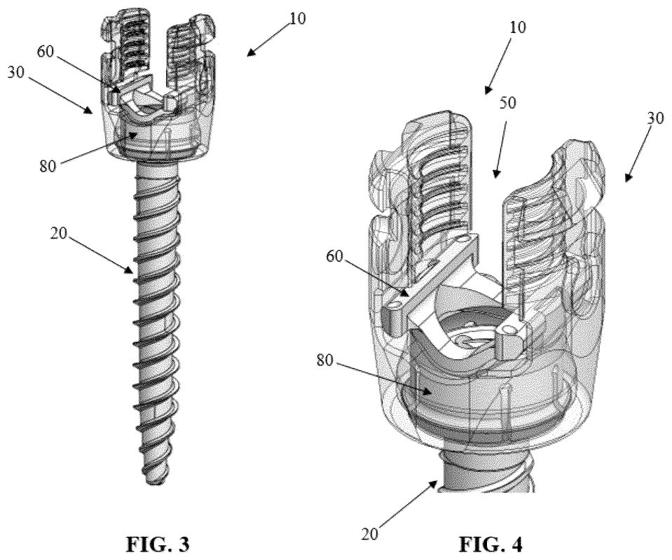

FIGS. 1-12 illustrate a polyaxial pedicle screw arrangement 10 in accordance with the present disclosure.

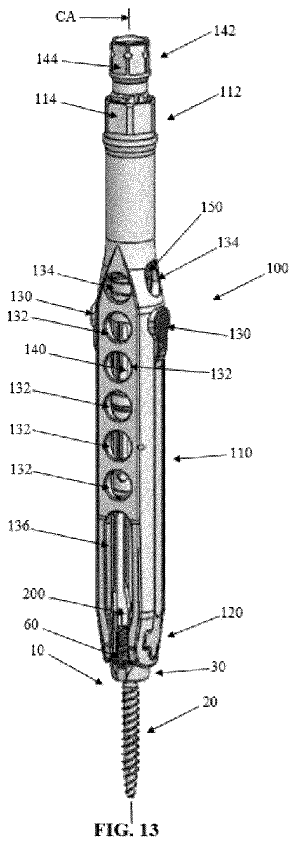

FIGS. 13-17 illustrate a medical instrument 100 that can be used with the polyaxial pedicle screw arrangement 10 in accordance with the present disclosure.

The present disclosure is direct to a medical device in the form of a polyaxial pedicle screw arrangement 10 for use in spinal implant applications. The polyaxial pedicle screw arrangement 10 can be configured to be temporarily locked in place during a surgical procedure. The temporary locking feature of the polyaxial pedicle screw arrangement 10 enables a user to perform one or more repositioning maneuvers of the polyaxial pedicle screw arrangement 10 during a surgical procedure (e.g., derotation, compression, distraction etc.). The provisional/temporary fastening arrangement of the polyaxial pedicle screw arrangement 10 can be configured to be activated and deactivated by the use of one or more medical instruments 100 that are configured to engage the polyaxial pedicle screw arrangement 10 in a particular region of the polyaxial pedicle screw arrangement 10 to cause the provisional/temporary fastening arrangement to lock the polyaxial pedicle screw in a current orientation. The medical instrument 100 can be configured to engage the polyaxial pedicle screw arrangement in a particular region of the polyaxial pedicle screw arrangement and optionally apply pressure to the provisional/temporary fastening arrangement and/or to cause movement of one or more components of the provisional/temporary fastening arrangement to move to the locked position and cause the polyaxial pedicle screw arrangement 10 to be locked in its current orientation. The polyaxial pedicle screw arrangement 10 can optionally be configured such that when the medical instrument 100 is partially or fully disengaged from the provisional/temporary fastening arrangement and/or causes movement of one or more components of the provisional/temporary fastening arrangement, the provisional/temporary fastening arrangement 10 is caused to move to an unlocked position which thereby enables the polyaxial pedicle screw arrangement 10 to move in various axial orientations. The medical instrument can be configured to cause a pressure and/or a compressive force on one or more portions of the provisional/temporary fastening arrangement to cause movement of one or more components provisional/temporary fastening arrangement to thereby convert (e.g., temporarily convert or permanently convert) the polyaxial pedicle screw arrangement into a monoaxial-behaving screw. The medical instrument can be configured to cause a pressure and/or a compressive force on two or more regions or portions of the provisional/temporary fastening arrangement (e.g., 2-8 regions or portions of the provisional/temporary fastening arrangement and all values and ranges therebetween) to cause movement of one or more components provisional/temporary fastening arrangement to thereby convert (e.g., temporarily convert or permanently convert) the polyaxial pedicle screw arrangement 10 in to a monoaxial-behaving screw.

In one non-limiting configuration the polyaxial pedicle screw arrangement 10 includes a tulip arrangement 30, an upper pressure cap 60, a lower pressure cap/flexible collet 80, and a pedicle screw 20. In one non-limiting embodiment, the one or more medical instruments 100 are configured to cause a pressure and/or a compressive force on one or more regions or portions of the upper pressure cap 60 (e.g., 1-8 regions or portions of the upper pressure cap, 2 regions of the upper pressure cap, 4 regions of the upper pressure cap, etc.) and/or the lower pressure cap/flexible collet 80 to cause movement of one or more regions of the upper pressure cap 60 and/or the lower pressure cap/flexible collet 80 to thereby convert (e.g., temporarily convert or permanently convert) the polyaxial pedicle screw arrangement into a monoaxial-behaving screw. In another and/or alternative non-limiting configuration, the one or more medical instruments 100 are configured to cause a pressure and/or a compressive force on one or more corner regions of the upper pressure cap 60 (e.g., 2-8 corner regions of the upper pressure cap, 2 corner regions of the upper pressure cap, 4 corner regions of the upper pressure cap, etc.) to cause movement of one or more corner regions of the upper pressure cap 60 to thereby convert (e.g., temporarily convert or permanently convert) the polyaxial pedicle screw arrangement 10 in to a monoaxial-behaving screw.

The pedicle screw 20 can be configured for used in a variety of bones such as, but not limited to, spinal bones (e.g., cervical spine (C1-C7), thoracic spine (T1-T12), lumbar spine (L1-L5), sacral spine (S1-S5), tailbone). The pedicle screw 20 includes a head portion 22 and a body portion 24. The head portion 22 generally has a maximum cross-sectional area that is greater than a maximum cross-sectional area of the body portion 24. The head portion 22 may or may not include external threads. The body portion 24 generally includes threads 26; however, this is not required. The threads 26 (when used) are generally spiral shaped; however, this is not required. For example, the body portion 24 can be fully threaded, partially threaded, comprise a spiral or helical blade, and/or may comprise one or more tacks, deployable talons, expanding elements, or so forth. As can be appreciated, the body 24 of the pedicle screw 20 can be a peg or pin shape. The threads 26 on the head portion 22 and/or body portion 24 of the screw (when used) are non-limiting (e.g., right-hand threads, left-hand threads, taper threads, “V” shape threads, metric threads, British threads, seller threads, square threads, acme threads, buttress threads, knuckle threads, worm threads, single and multi-threads). The end region of the body portion 24 can optionally include a self-tapping or self-drilling tip; however, this is not required. The shape of the head portion 22 is non-limiting. In one non-limiting embodiment, the cross-sectional shape of top region of the head portion is generally circular shaped; however, other shapes can be used. The head portion can optionally include a cavity 28 to facilitate in inserting the orthopedic screw into a bone. The configuration of the cavity 28 (when used) is non-limiting. Generally, the cavity is specially shaped to receive a tool to rotate and/or otherwise cause the pedicle screw to be inserted into a bone. Non-limiting shapes that can be used in the cavity 28 include one or more dimples, ridges, bumps, textured areas, star shaped, polygonal shaped, or any other surface or shape. As can be appreciated, the cavity 28 can optionally include a threaded inner surface.

The tulip 30 includes a body 32 that includes a top portion 34 and a bottom portion 36. The body 32 generally has a circular cross-sectional shape; however, other cross-sectional shapes can be used (e.g., oval, triangular, square, rectangular, polygonal, etc.). In one non-limiting embodiment, the tulip 30 is configured to facilitate in the connection to an orthopedic device OD (e.g., spinal rod, etc.). The outer surface of body 32 can optionally include one or more surface structures 38 (e.g., slot, cavity, rib, depression, etc.) that is used to facilitate in the medical instrument 100 releasably engaging the outer surface of the body 32 of tulip 30. The number, size, shape and configuration of the one or more surface structures 38 are non-limiting. The top portion 34 of the body 32 includes of the tulip 30 optionally includes one or more side guide surfaces 39 that are configured to facilitate in the guiding and positioning of engagement legs 200 of the inner body 140 of the medical instrument 100 during movement of the inner body 140 relative to tulip 30. The top portion 34 of the body 32 includes a top cavity 40. The internal surface of the top cavity can include a connection surface 42 such as, but not limited to, a threaded surface. The cross-sectional shape of the top cavity 40 is generally circular; however, other shapes can be used. The longitudinal length of the top cavity 40 is generally 50-100% (and all values and ranges therebetween) of the longitudinal length of the top portion 34. The top cavity 40 includes a top opening that is configured to allow an instrument (e.g., screwdriver, locking tool, etc.) to be inserted into the top cavity 40 of the top portion of the body to enable the instrument to access a locking device LD (e.g., locking screw, etc.) and manipulate (e.g., turn, push, move, etc.) the locking device LD. The side of the top portion 34 includes first and second top side openings 44, 46 that are configured to receive an orthopedic device OD (e.g., rod, etc.). In one non-limiting embodiment, the top cavity 40 includes a connection arrangement in the form of a threaded connection arrangement 42 that is configured to receive a locking device LD (e.g., threaded lock screw, etc.) to secure an orthopedic device OD (e.g., rod, etc.) in top cavity 40. The first and second top side openings 44, 46 are configured to be positioned on opposite sides of the top cavity 40 and extend upwardly to the top opening of the top cavity 40 of the top portion 34. The first and second top side openings 44, 46 form two or more upwardly extending arms in the top portion 34. The longitudinal length of the first and second top side openings 44, 46 generally extends 10-100% (and all values and ranges therebetween) of the longitudinal length of the top portion 34. In one non-limiting arrangement, after an orthopedic device OD (e.g., rod, etc.) is inserted through and/or into the first and second top side openings 44, 46, a locking device LD (e.g., threaded screw, etc. can be inserted into the top opening in the top cavity 40 and connected thereto (e.g., the locking screw can be threaded on the threaded surface in the cavity of the top portion, etc.) to secure and lock the orthopedic device OD (e.g., rod, etc.) in position relative to the body 32 of the tulip 30. As can be appreciated, many different arrangements can be used to secure the orthopedic device OD (e.g., rod, etc.) to the top portion 34 of the body 32 of the tulip 30. In another non-limiting embodiment, the first and second top side openings 44, 46 can optionally have a generally U-shaped configuration; however; other shapes can be used (e.g., triangular, square, rectangular, polygonal, oval, etc.). The size and shape of the first and second top side openings 44, 46 is generally the same; however, this is not required. The top portion 34 can include first and second side slots 48, 49 that are configured to receive a portion of an upper pressure cap 60 as will be discussed in more detail below. The bottom portion 36 of the body 32 generally has a longitudinal length that is 30-70% (and all values and ranges therebetween) of the longitudinal length of the top portion 34 of the body 32; however, this is not required. The body 32 generally includes a mid-opening 50 that is positioned fully in the top portion, fully in the bottom portion, or partially in the top and bottom portion of the body 32 of the tulip 30. The mid-opening 50 is generally positioned about the central axis of the body 32 of the tulip. The cross-sectional shape of the mid-opening 50 is generally circular; however, this is not required. Generally, the longitudinal length of the mid-opening as measured along the longitudinal axis of the body 32 is generally 5-40% (and all values and ranges therebetween) of the longitudinal length of the body 32. The maximum diameter of the mid-opening 50 or the maximum cross-sectional area of the mid-opening 50 is generally 50-90% (and all values and ranges therebetween) of the maximum diameter of the body 32 or the maximum cross-sectional area of the body 32 that contains the mid-opening 50. The bottom portion 36 of the body 32 includes a bottom cavity 52 that is located below the mid-opening 50 of the body 32. The size and/or shape of the bottom cavity 52 of the bottom portion 36 can be the same or different from the top cavity 40 in the top portion 34; however, this is not required. In one non-limiting embodiment, the cross-sectional shape of the bottom cavity 50 is generally circular; however, other shapes can be used. The longitudinal length of the bottom cavity 52 is generally 50-100% (and all values and ranges therebetween) of the longitudinal length of the bottom portion 36. The internal surface of the bottom cavity 52 can optionally include a connection surface (e.g., threaded surface, etc.) or be a smooth surface. The longitudinal length of the bottom cavity 52 is generally 50-100% (and all values and ranges therebetween) of the longitudinal length of the bottom portion 36. The bottom cavity 52 is shaped and sized to as to partially or fully telescopically receive the lower pressure cap/flexible collet 80 as will be further discussed below.

The upper pressure cap 60 and the lower pressure cap/flexible cap 80 are used to temporarily locked in place a polyaxial pedicle screw arrangement in position during a surgical procedure. The upper pressure cap 60 is configured to be positioned above the lower pressure cap/flexible cap 80 and is configured to engage the lower pressure cap/flexible cap 80. The upper pressure cap 60 includes side flanges 62, 64 that are configured to slide into first and second side slots 48, 49 of top portion 34 of tulip 30. When the side flanges 62, 64 are positioned in first and second side slots 48, 49 of top portion 34 of tulip 30, the upper pressure cap 60 is limited in the distance that the upper pressure cap 60 can vertically move upwardly toward the top portion 34. Generally, when the side flanges 62, 64 are positioned first and second side slots 48, 49 of top portion 34 of tulip 30, the upper pressure cap 60 is prevented from vertically moving upwardly no more than 25% (e.g., 0.1-25% and all values and ranges therebetween) of the central axial length of the top portion 34 of the tulip 30. The first and second side slots 48, 49 are configured to allow some upward and downward movement of the side flanges 62, 64 within first and second side slots 48, 49 to facilitate in the locking of the pedicle screw relative to the lower pressure cap/flexible cap 80 as will be discussed in more device below. Each of side flanges 62, 64 includes one or more side slots 66, 68 (e.g., 1-4 side slots and all values and ranges therebetween) that are configured to receive a portion of lower pressure cap/flexible cap 80. The upper pressure cap 60 include a central opening 70. Generally, the cross-sectional area or diameter of the central opening 70 is less than the maximum cross-sectional area or maximum diameter of head portion 22 of the pedicle screw 20 such that the head portion 22 is unable to fully pass through central opening 70. The central region of the upper pressure cap 60 that is located between side flanges 62, 64 can optionally curve downwardly; however, this is not required. The upper pressure cap 60 includes one or more engagement areas 72 (e.g., 1-8 engagement areas and all values and ranges therebetween) that are configured to be engaged by a medical instrument 100. The one or more engagement area 72 can be optionally located on at least a portion or all of the side flanges 62, 64 of the upper pressure cap 60. In one non-limiting arrangement, an engagement area 72 is located on a top surface of each end region of each of side flanges 62, 64. As will be discussed in more detail below, the upper pressure cap 60 is configured to be engaged by a medical instrument 100 at the one or more engagement areas 72, and pressure that is applied by the medical instrument 100 on the one or more engagement areas 72 causes movement of the lower pressure cap/flexible cap 80, that is located below the upper pressure cap 60, which in turn causes the polyaxial pedicle screw arrangement 10 to be temporarily locked in in position. The one or more engagement areas 72 can optionally include a recessed region that is configured to receive a portion of the medical instrument 100. In an alternative arrangement, the one or more engagement areas 72 are in the form of openings that pass fully through the upper pressure cap 60. In such an arrangement, a portion of the medical instrument 100 is configured to fully pass though the opening in one or more of the engagement areas 72 and thereafter directly contact the lower pressure cap/flexible cap 80, that is located below the upper pressure cap 60, and cause movement of the lower pressure cap/flexible cap 80, which in turn causes the polyaxial pedicle screw arrangement 10 to be temporarily locked in in position. As can be appreciated, a combination of these two arrangements or other alternative arrangement can be used to cause movement of the lower pressure cap/flexible cap 80 by the medical instrument 100.

The lower pressure cap/flexible cap 80 includes a body 82 that has a central axial cavity 84. The outer surface of body 82 can have a circular cross-sectional shape; however, this is not required. Cavity 84 generally has a circular cross-sectional shape. The cross-sectional area of cavity 84 can be constant or can vary along the central axis of cavity 84. The size of cavity 84 is selected such that the maximum diameter of maximum cross-sectional area of the head portion 22 of the pedicle screw 20 can pass through the bottom region of cavity 84. In one non-limiting arrangement, the size of cavity 84 is selected such that the maximum diameter of maximum cross-sectional area of the head portion 22 of the pedicle screw 20 can pass through 30-100 % (and all values and ranges therebetween) of the longitudinal length of cavity 84. The top surface 86 of body 82 can optionally have a curved profile that partially recesses in body 82. The profile of top surface 86 of body 82 can be such that is closely mates with a portion of the profile of the bottom surface of the upper pressure cap 60; however, this is not required. One or more position flanges 88, 90 (e.g., 1-8 position flanges and all values and ranges therebetween) extend upwardly from the top surface 86 of body 82. A portion or all of one of position flanges 88, 90 is configured to at least partially enter into one of side slots 66, 68 of side flanges 62, 64 of upper pressure cap 60 when the polyaxial pedicle screw arrangement 10 is fully assembled. The positioning of the position flanges 88, 90 into side slots 66, 68 of side flanges 62, 64 of upper pressure cap 60 a) facilitates in proper positioning of the upper pressure cap 60 relative to the lower pressure cap/flexible cap 80, and/or b) inhibits or prevents undesired movement of the lower pressure cap/flexible cap 80 about the central axis of tulip 30 (e.g., limits rotational movement of the lower pressure cap/flexible collet about a central axis of the tulip) when the polyaxial pedicle screw arrangement 10 is fully assembled. Furthermore, the upper pressure cap 60 inhibits or prevents upward movement of the lower pressure cap/flexible cap 80 along the central axis of tulip 30 when the polyaxial pedicle screw arrangement 10 is fully assembled. The wall of the lower portion of body 82 optionally includes one or more slots 92 (e.g., 1-10 slots and all values and ranges therebetween) that extend from the base of the body 82 to a point that is spaced from an upper edge of body 82. Generally, each of slots 92 extends 40-95% (and all values and ranges therebetween) the longitudinal length of body 82. The slots 92 can be optionally oriented on body 82 such that adjacently positioned slots are equally spaced form one another; however, this is not required. The one or more slots 92 are configured to enable the wall of the lower portion of body 82 to be compressed and to reduce in cross-sectional area. In one non-limiting arrangement the cross-sectional area or diameter of the lower portion or bottom end of bottom cavity 52 in body 32 of tulip 30 is less than a maximum cross-sectional area or maximum diameter of the bottom portion of body 82 of lower pressure cap/flexible cap 80. Such reduction in cross-sectional area or diameter can be obtained by a) the bottom end portion 54 of the bottom portion 36 of body 32 tapering inwardly, b) the changing of the thickness of the wall of the bottom portion 36, and/or c) the changing of the inner and/or outer diameter of the wall of the bottom portion 36. In one non-limiting arrangement, the thickness of the bottom portion 36 of the body is varied such that the bottom end portion 54 forms a bottom opening 56 into bottom cavity 52 that has a maximum cross-sectional area or maximum diameter that is less than a portion of the bottom cavity that is located above the bottom end portion 54. Due to the cross-sectional area or diameter of the lower portion or bottom end of bottom cavity 52 in body 32 of tulip 30 being less than a maximum cross-sectional area or maximum diameter of the bottom portion of body 82 of lower pressure cap/flexible cap 80, when the body 82 of lower pressure cap/flexible cap 80 is inserted into bottom cavity 52 in body 32 of tulip 30, the one or more slots 92 enable the wall of the lower portion of body 82 to be compressed and to reduce in cross-sectional area so as to pass through the lower portion or bottom end 54 of bottom cavity 52 in body 32 of tulip 30. Once the bottom portion of body 82 of lower pressure cap/flexible cap 80 pass by the lower portion or bottom end 54 of bottom cavity 52 in body 32 of tulip 30, the cross-sectional area or diameter of the bottom cavity increases thereby allowing the bottom portion of body 82 of lower pressure cap/flexible cap 80 to reexpand or spring or flex back to the original or near original (e.g., 90-99.999% of original and all values and ranges therebetween) cross-sectional area or diameter or position. Such reexpansion, spring back or flex back of the bottom portion of body 82 of lower pressure cap/flexible cap 80 inhibits or prevents that lower pressure cap/flexible cap 80 from exiting through the lower portion or bottom end 54 of bottom cavity 52 in body 32 of tulip 30 without having to first applying a substantial force to the lower pressure cap/flexible cap 80 to cause the lower portion of body 82 to be compressed. The bottom region or end of body 82 of lower pressure cap/flexible cap 80 can optionally include a tapered base flange 94. The tapered base flange includes a tapered bottom region and optionally a tapered top region. The tapered top region, when used, facilitates in the insertion of the lower pressure cap/flexible cap 80 into the bottom cavity 52 in body 32 of tulip 30 during assembly of the polyaxial pedicle screw arrangement 10. The tapered bottom region is configured to engage the lower portion or bottom end 54 of bottom cavity 52 in body 32 of tulip 30 when the lower pressure cap/flexible cap 80 is caused to move downwardly within the bottom cavity 52 when a downwardly force from upper pressure cap 60 is applied to the top surface of body 82 of lower pressure cap/flexible cap 80. The upper pressure cap 60 can be caused to produce such downward force on lower pressure cap/flexible cap 80 when a medical instrument 100 applies a downward force onto upper pressure cap 60 as discussed above. When the polyaxial pedicle screw arrangement 10 is fully assembled, the downward movement of the lower pressure cap/flexible cap 80 causes the outer surface of the wall or base flange 94 of body 82 to engage the lower portion or bottom end 54 of bottom cavity 52 in body 32 of tulip 30 and thereby cause the cross-sectional area or diameter of the lower portion of body 82 to be reduces, and such reduction in cross-sectional area or diameter of the lower portion of body 82 causes the cross-sectional area or diameter of cavity 84 to reduce and compress against the head portion 22 of the pedicle screw 20 and thereby prevent movement of the head portion 22 of the pedicle screw 20 within cavity 84. Such prevention of movement of the head portion 22 of the pedicle screw 20 within cavity 84 causes the polyaxial pedicle screw arrangement 10 to be temporarily locked in in position, thereby temporarily converting the polyaxial pedicle screw arrangement into a monoaxial-behaving pedicle screw arrangement.

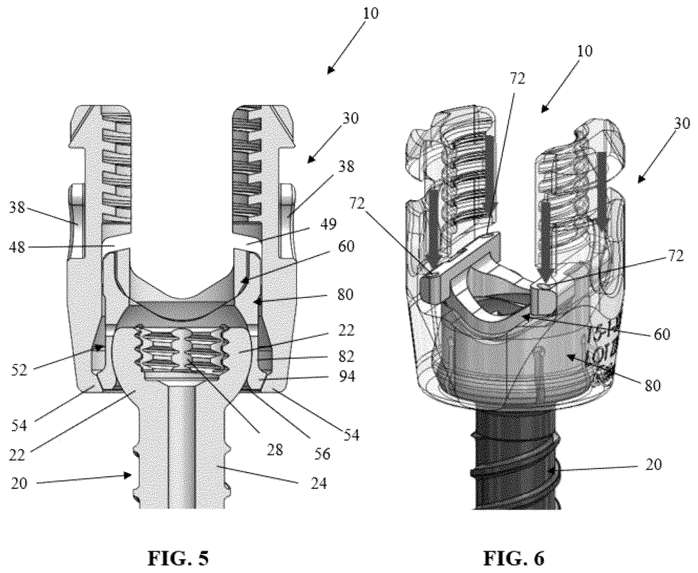

As discussed above, the downward force can be applied by one or more medical instruments 100. As represented in FIGS. 5 and 6, the downward force on the upper pressure cap 60 causes the side flanges 62, 64 of upper pressure cap 60 to move downwardly in first and second side slots 48, 49 of tulip 30. The size of the first and second side slots 48, 49 of tulip 30 limit the distance of downward movement of upper pressure cap 60 in the tulip 30. The downward movement of the upper pressure cap 60 causes the lower pressure cap/flexible cap 80 to also move downwardly in tulip 30. As the lower pressure cap/flexible cap 80 moves downwardly, the outer surface of the wall or base flange 94 of body 82 engages the lower portion or bottom end 54 of bottom cavity 52 in body 32 of tulip 30 and is cause to bend inwardly, which in turn causes the cross-sectional area or diameter of the lower portion of body 82 to be reduced, and such reduction in cross-sectional area or diameter of the lower portion of body 82 causes the cross-sectional area or diameter of cavity 84 to reduce and compress against the head portion 22 of the pedicle screw 20 and thereby prevent movement of the head portion 22 of the pedicle screw 20 within cavity 84. Such prevention of movement of the head portion 22 of the pedicle screw 20 within cavity 84 causes the polyaxial pedicle screw arrangement 10 to be temporarily locked in in position, thereby temporarily converting the polyaxial pedicle screw arrangement into a monoaxial-behaving pedicle screw arrangement. As can be appreciated, when the downward force is removed from the upper pressure cap 60, the upper pressure cap 60 can move upwardly in first and second side slots 48, 49 of tulip 30, thus also allowing lower pressure cap/flexible cap 80 to also move upwardly in tulip 30, which upward movement of lower pressure cap/flexible cap 80 allow the walls of the lower portion of body 82 to expand and once again allow movement of the head portion 22 of the pedicle screw 20 relative to the lower pressure cap/flexible cap 80.

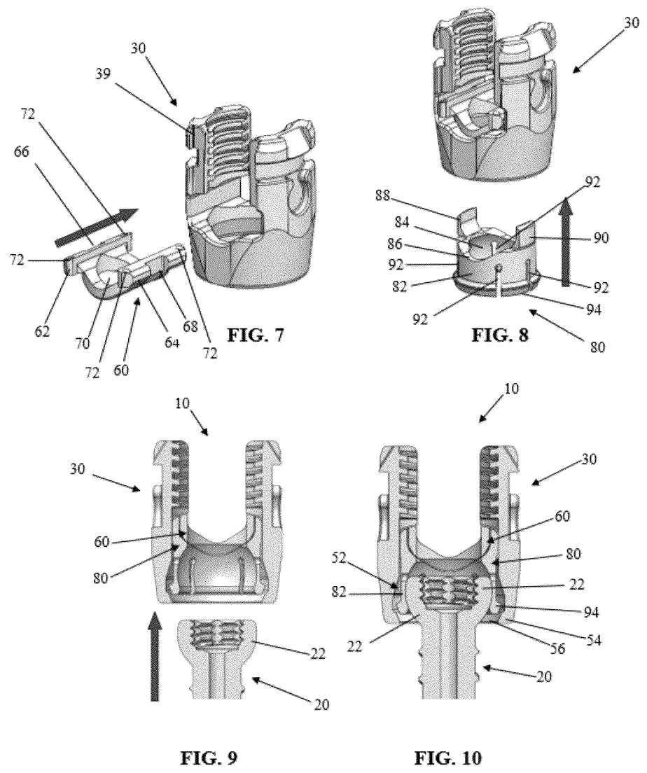

FIG. 7 illustrates the upper pressure cap 60 and the tulip 30 and how the upper pressure cap 60 is inserted into the tulip 30.

FIG. 8 illustrates the lower pressure cap/flexible cap 80 and the tulip 30 and how the lower pressure cap/flexible cap 80 is inserted into the tulip 30.

FIG. 9 illustrates the pedicle screw 20 and the tulip 30 and how the pedicle screw 20 is inserted into the tulip 30 when the lower pressure cap/flexible cap 80 has also been inserted into the tulip 30. When the lower pressure cap/flexible cap 80 is positioned in its upper portion as illustrated in FIG. 9, the head portion 22 of the pedicle screw 20 is able to be inserted into cavity 84 of body 82 of the lower pressure cap/flexible cap 80. In such position, the portion of the body 82 and the tapered base flange 94 that are adjacent to one or more slots 92 94 can flex outwardly a sufficient distance within bottom cavity 52 of tulip 30 to enable the head portion 22 of the pedicle screw 20 to fully pass by tapered base flange 94 and into cavity 84 of body 82 of the lower pressure cap/flexible cap 80. Once the head portion 22 of the pedicle screw 20 passes by tapered base flange 94, the portion of the body 82 and the tapered base flange 94 that are adjacent to one or more slots 92 94 can flex back to their original positions. The bottom cavity 52 of tulip 30 can be configured such that only when the lower pressure cap/flexible cap 80 is positioned in its upper portion as illustrated in FIG. 9 can the body 82 and the tapered base flange 94 that are adjacent to one or more slots 92 94 flex outwardly a sufficient distance within bottom cavity 52 of tulip 30 to enable the head portion 22 of the pedicle screw 20 to fully pass by tapered base flange 94 and into cavity 84 of body 82 of the lower pressure cap/flexible cap 80.

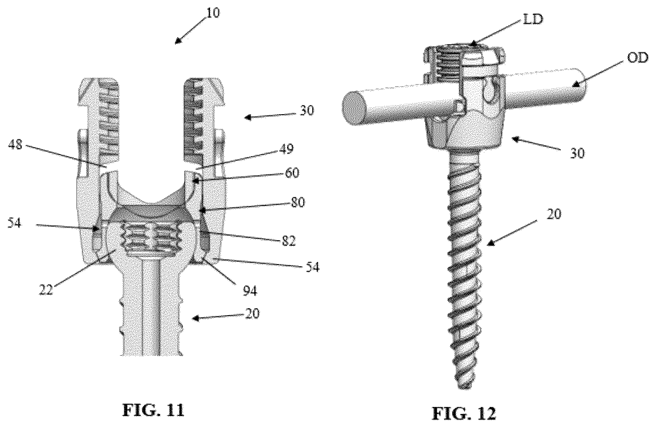

FIG. 10 illustrates the polyaxial pedicle screw arrangement 10 prior to downward force being applied to the upper pressure cap 60 and wherein the head portion 22 of the pedicle screw 20 is able to move relative to the lower pressure cap/flexible cap 80. FIG. 11 illustrates the polyaxial pedicle screw arrangement 10 after a downward force has been applied to the upper pressure cap 60 which has caused both the upper pressure cap 60 and the lower pressure cap/flexible cap 80 to move downwardly in tulip 30, and wherein the head portion 22 of the pedicle screw 20 is temporarily locked in position relative to the lower pressure cap/flexible cap 80.

FIG. 12 illustrates an orthopedic device OD in the form of a rod that is secured to tulip 30 by a locking device LD that is in the form of a screw that is threaded to the tulip 30. A pedicle screw 20 is connected to the bottom portion of the tulip 30.

FIG. 13 illustrates a non-limiting medical instrument 100 that can be used with the polyaxial pedicle screw arrangement 10. The medical instrument 100 includes an outer body 110 and an inner body 140. The inner body 140 is configured to be rotatably positioned in the outer body 110 and rotatable about a central longitudinal axis CA of the medical instrument 100. A top portion 142 of the inner body 140 extends upwardly from a top portion 112 of the outer body. The top portion 142 of the inner body 140 optionally includes one or more engagement members 144 that are configured to be engaged by a medical tool (not shown) to facilitate in the rotation of the inner body 140 relative to the outer body 110 of the medical instrument 100. The size, shape and configuration of the one or more engagement members 144 are non-limiting. As illustrated in FIG. 13, the top portion 142 of the inner body 140 has a non-limiting hexagonal cross-sectional shape with six flat faced engagement members 144. The bottom portion 120 of outer body 110 is configured to releasably engage an outer surface of tulip 30. The outer surface of the outer body 110 can optionally include one or more gripping regions 130 that can be used by a user to facilitate in the gripping of the medical instrument 100 during use. The size, shape and configuration of the one or more gripping regions 130 are non-limiting. The outer body 110 can optionally include one or more side openings 132 that can be used to a) reduce the weight of the outer body 110, and/or b) enable a user to view the movement of one or more portions of the inner body 140 within the outer body 110. The size, shape and/or configuration of the one or more side openings 132 are non-limiting. The outer body 110 can optionally include one or more upper side openings 134 that can be used to a) reduce the weight of the outer body 110, and/or b) enable a user to view the distance of longitudinal movement of the inner body 140 within outer body 110. The size, shape and/or configuration of the one or more upper side openings 134 are non-limiting. The inner body 140 can optionally include numbering 150 or other types of designations that inform a user the distance of longitudinal movement of the inner body 140 within outer body 110 during use of the medical device 100. The outer body 110 can optionally include one or more bottom slots or openings 136 that can be used to a) reduce the weight of the outer body 110, b) enable a used to view the movement of one or more portions of the inner body 140 within the outer body 110, and/or c) enable a user to view the positioning of the engagement legs 200 relative to the engagement areas 72 of the upper pressure cap 60. The size, shape and/or configuration of the one or more bottom slots or openings 132 are non-limiting. A top portion 112 of the outer body 110 optionally includes one or more gripping members 114 that are configured to be engaged by a medical tool (not shown) to a) maintain the position of the outer body during a surgical procedure, b) rotate the outer body during a surgical procedure, and/or c) maintain the position of the outer body during rotation of the top portion 142 of the inner body 140. The size, shape and configuration of the one or more gripping members 114 are non-limiting. As illustrated in FIG. 13, the top portion 112 of the outer body 110 has a non-limiting hexagonal cross-sectional shape with six flat faced gripping members 114.

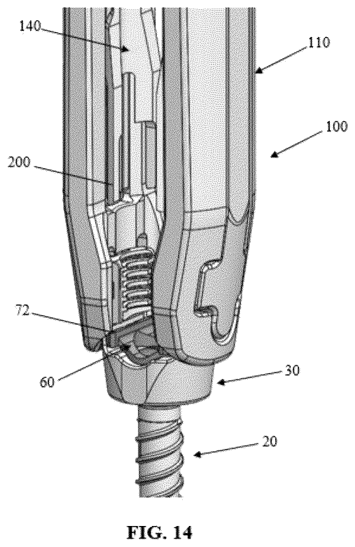

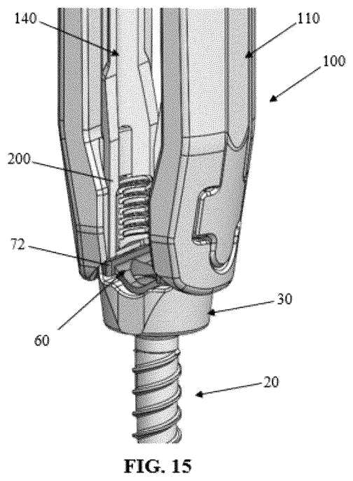

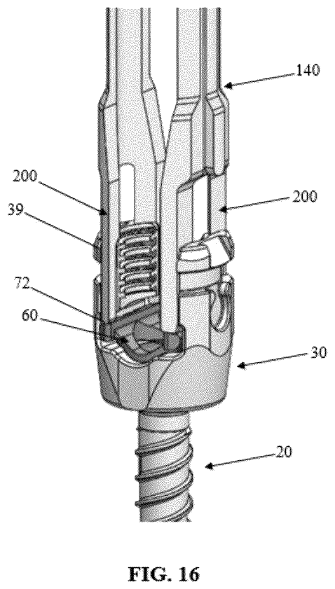

FIG. 14 is an enlarged view of the bottom portion of the medical instrument 100 that is in engagement with the tulip 30 of polyaxial pedicle screw arrangement 10 and wherein the bottom engagement legs 200 of the inner body 140 are spaced upwardly from engagement areas 72 of the upper pressure cap 60. FIG. 15 is an enlarged view of the bottom portion of the medical instrument 100 that is in engagement with the tulip 30 of polyaxial pedicle screw arrangement 10 and wherein the bottom engagement legs 200 of the inner body 140 are engaged with the engagement areas 72 of the upper pressure cap 60. FIG. 16 is a similar view as in FIG. 15 except that the outer body 110 of the medical instrument 100 is not shown.

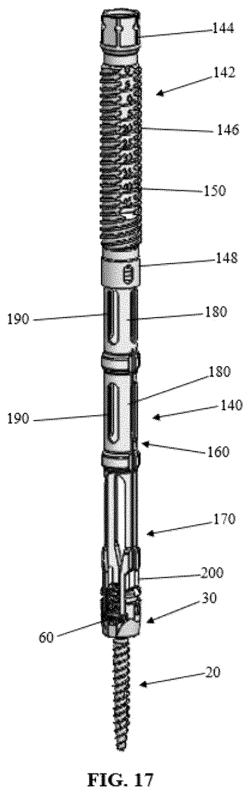

FIG. 17 is an illustration of inner body 140 of the medical instrument 100. The top portion 142 of the inner body 140 optionally includes one or more engagement members 144 that are configured to be engaged by a medical tool (not shown) to facilitate in the rotation of the inner body 140 relative to the outer body 110 of the medical instrument 100. The top portion 142 of the inner body 140 is illustrated as having a non-limiting hexagonal cross-sectional shape with six flat faced engagement members 144; however, it will be appreciated that other configurations of the top portion 142 can be used. Positioned below the top portion 142 is a threaded region 146. The threaded region 146 is configured to engage a threaded surface on the top portion 112 of the outer body 110 of the medical device 100. When the inner body 140 is rotated relative to the outer body 110 about the central axis CA of the medical instrument, the threaded region 146 on the inner body 140 and the threaded surface on the top portion 112 of the outer body 110 cause the inner body 140 to move upwardly and downwardly relative to the outer body 110 depending on the direction of rotation of the inner body 140. As the inner body 140 moves upwardly and/or downwardly relative to the outer body, a user can view the degree of movement of the inner body by viewing the numbering 150 or other types of designations (e.g., A, B, C; 1, 2, 3, etc.) on the top portion 142 of the inner body 140 via upper side openings 134 in the outer body 110. The bottom region 148 of the top portion 142 includes a coupling arrangement that is configured to secure the top portion of the inner body 140 to the bottom portion 160 of the inner body 140. The bottom region 170 of the bottom portion 160 includes a plurality of engagement legs 200. The bottom portion 160 can optionally include one or more extension sections 180 that are configured to be coupled together and used to obtain the desired length of the inner body 140. The one or more extension sections can optionally include one or more slots 190. The one or more slots 190 can be used to reduce the weight of the inner body and/or reduce the amount of material used in the inner body 140. As illustrated in FIG. 17, the inner body includes two extension sections 180. The first extension section 180 is connected at one end to the top portion 142 of the inner body 140 and the other end of the first top extension section 180 is connected to the top end of the second extension section 180. The other end of the second extension section 180 is connected to the bottom region or bottom section 170 of the inner body 140. The arrangement used to couple the extension sections together and/or to other portions of the inner body 140 is non-limiting (e.g., threaded connection, slot & lock connection, adhesive, etc.).

When an orthopedic device OD (e.g., rod, etc.) is inserted into first and second top side openings 44, 46 of cavity 40 in the top portion 34 of tulip 30 and thereafter secured in cavity 40 by a locking device LD (e.g., threaded lock screw, etc.) or other type of connector that is connected to connection arrangement 42, the orthopedic device is caused to press downwardly and cause a downward force to be applied on upper pressure cap 60. Such downward force on the upper pressure cap 60 causes the lower pressure cap/flexible cap 80 to also move downwardly in tulip 30. As the lower pressure cap/flexible cap 80 moves downwardly, the outer surface of the wall or base flange 94 of body 82 engages the lower portion or bottom end 54 of bottom cavity 52 in body 32 of tulip 30 and is cause to bend inwardly, which in turn causes the cross-sectional area or diameter of the lower portion of body 82 to be reduced, and such reduction in cross-sectional area or diameter of the lower portion of body 82 causes the cross-sectional area or diameter of cavity 84 to reduce and compress against the head portion 22 of the pedicle screw 20 and thereby prevent movement of the head portion 22 of the pedicle screw 20 within cavity 84. Such prevention of movement of the head portion 22 of the pedicle screw 20 within cavity 84 causes the polyaxial pedicle screw arrangement 10 to be locked in in position, thereby converting the polyaxial pedicle screw arrangement into a monoaxial-behaving pedicle screw arrangement.

One or more of the components can be formed of a variety of materials. In one non-limiting embodiment, the metal portion of the polyaxial pedicle screw arrangement 10 is partially (e.g. 1-99.999 wt. % and all values and ranges therebetween) or fully formed of a metal material that includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, or m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) rhenium (e.g., 5-99 awt. % rhenium and all values and ranges therebetween). The metal alloy used to partially or fully form the polyaxial pedicle screw arrangement 10 can be nitrided; however, this is not required.

The polyaxial pedicle screw arrangement 10 can include, contain, and/or be coated with one or more agents that facilitate in the success of the polyaxial pedicle screw arrangement 10 and/or treated area.

The polyaxial pedicle screw arrangement 10 can include a marker material.

The polyaxial pedicle screw arrangement 10 or one or more regions of the polyaxial pedicle screw arrangement 10 can be constructed by use of one or more microelectromechanical manufacturing (MEMS) techniques (e.g., micro-machining, laser micro-machining, laser micro-machining, micro-molding, etc.); however, other or additional manufacturing techniques can be used.

One or more portions of metal portion of the polyaxial pedicle screw arrangement 10 can be partially (e.g., 1% to 99.99% and all values and ranges therebetween) or fully be coated with an enhancement layer to improve one or more properties of the polyaxial pedicle screw arrangement 10 (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement layer forms a surface that is less of an irritant to cell about the coated surface after the polyaxial pedicle screw arrangement 10 is implanted, reduces the rate to which cells grown on coated surface after the polyaxial pedicle screw arrangement 10 is implanted, reduce rate to which movable components in the polyaxial pedicle screw arrangement 10 fail to properly operate after orthopedic device is implanted, etc.).

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Office and any readers of this application and any resulting patent in interpreting the claims appended hereto, Applicant does not intend any of the appended claims or claim elements to invoke 35 U.S.C. 112(f) unless the words “means for” or “step for” are explicitly used in the particular claim.

1. A polyaxial pedicle screw arrangement that can be temporarily locked in position during a surgical procedure; said polyaxial pedicle screw arrangement comprising a tulip, an upper pressure cap, a lower pressure cap/flexible collet, a locking device, and a pedicle screw; said upper pressure cap and/or said lower pressure cap/flexible collet are configured to move within said tulip when a force is applied to said upper pressure cap and/or said lower pressure cap/flexible collet by said locking device locking an orthopedic device in said tulip; said upper pressure cap separate component from said lower pressure cap/flexible collet; a lower portion of said upper pressure cap is configured to engage an upper portion of said lower pressure cap/flexible collet, and wherein said movement of said upper pressure cap and/or said lower pressure cap/flexible collet in at least one direction within said tulip is configured to inhibit or prevent movement of said pedicle screw relative to said upper pressure cap and/or said lower pressure cap/flexible collet.

2. The polyaxial pedicle screw arrangement as defined in claim 1, wherein said upper pressure cap and/or said lower pressure cap/flexible collet are configured to move upwardly and downwardly along a longitudinal axis of said tulip, and wherein downward movement of said upper pressure cap and/or said lower pressure cap/flexible collet within said tulip is configured to inhibit or prevent movement of said pedicle screw relative to said upper pressure cap and/or said lower pressure cap/flexible collet.

3. The polyaxial pedicle screw arrangement as defined in claim 1, wherein said upper pressure cap is positioned above said pedicle screw and does not engage said pedicle screw.

4. The polyaxial pedicle screw arrangement as defined in claim 1, wherein said pedicle screw includes a head portion and a body portion; said head portion has a maximum cross-sectional area that is greater than a maximum cross-sectional area of said body portion.

5. The polyaxial pedicle screw arrangement as defined in claim 1, wherein said tulip includes body that has a top portion and a bottom portion; said top portion including a top cavity; said bottom portion including a bottom cavity; said top portion including two top side openings configured to receive a portion of an orthopedic rod or other type of medical device; said two top side openings forming first and second upwardly extending arms; said top cavity including a top securing surface configured to receive locking device to entrap the orthopedic rod or other type of medical device in said two top side openings and between said locking device and said upper pressure cap, and while said lower pressure cap/flexible collet is spaced from the orthopedic rod or other type of medical device; said bottom cavity is configured to receive at least a portion of said lower pressure cap/flexible collet and/or said pedicle screw.

6. The polyaxial pedicle screw arrangement as defined in claim 5, wherein said top securing surface includes a threaded surface.

7. The polyaxial pedicle screw arrangement as defined in claim 5, wherein said body of said tulip includes a mid-opening that is positioned along a longitudinal axis of said body; said opening forming a passageway between the top and bottom cavities.

8. The polyaxial pedicle screw arrangement as defined in claim 7, wherein a maximum cross-sectional area of said mid-opening is less than a maximum cross-sectional area of said head portion of said pedicle screw.

9. The polyaxial pedicle screw arrangement as defined in claim 5, wherein said body of said tulip includes one or more side slots that are configured to receive a portion of said upper pressure cap; said one or more side slots are configured to limit upward and downward movement of said upper pressure cap along said longitudinal axis of said tulip when said polyaxial pedicle screw arrangement is fully assembled.

10. The polyaxial pedicle screw arrangement as defined in claim 1, wherein said lower pressure cap/flexible cap includes a body that has a body central axial cavity; a lower portion of said body central cavity has a cross-sectional area such that said head portion of said pedicle screw can at least partially pass into said body central axial cavity.

11. The polyaxial pedicle screw arrangement as defined in claim 1, wherein said upper pressure cap includes one or more side slots that are configured to receive a portion of said lower pressure cap/flexible collet; said one or more side slots of said upper pressure cap are configured to limit rotational movement of said lower pressure cap/flexible collet about a central longitudinal axis of said tulip and allow upward and downward movement along said central longitudinal axis of said tulip when said pedicle screw arrangement is fully assembled.

12. The polyaxial pedicle screw arrangement as defined in claim 11, wherein said lower pressure cap/flexible collet includes one or more position flanges that extend upwardly from said body of said lower pressure cap/flexible cap, and wherein said one or more position flanges are configured to be at least partially inserted in one or more side slots of said upper pressure cap when said pedicle screw arrangement is fully assembled.

13. The polyaxial pedicle screw arrangement as defined in claim 10, wherein said body of said lower pressure cap/flexible collet includes one or more slots that extend from a base of said body to a point that is spaced from an upper edge of said body; said one or more slots are configured to enable a lower portion of said body to be compressed together and to reduce a cross-sectional area of said lower portion of said body when a downward force is applied to said lower pressure cap/flexible collet.

14. A medical instrument for use with said polyaxial pedicle screw arrangement as defined in claim 1, said medical instrument includes one or more engagement legs that are configured to engage a portion of said upper pressure cap and/or said lower pressure cap/flexible collet to cause movement of said upper pressure cap and/or said lower pressure cap/flexible collet.

15. The medical instrument as defined in claim 14, wherein said one or more engagement legs are configured to engage one or more engagement areas located on a top surface of said upper pressure cap; each of said one or more engagement areas is configured to telescopically receive a portion of one of said engagement legs.

16. The medical instrument as defined in claim 14, wherein said medical instrument includes an outer body and an inner body; said inner body is configured to be rotatably positioned in said outer body and be rotatable about a central longitudinal axis of said medical instrument; said inner body is configured to move along a central longitudinal axis of said outer body; a top portion of said inner body extends upwardly from a top portion of said outer body; a top portion of said inner body includes one or more engagement members that are configured to be engaged by a medical tool to facilitate in rotation of said inner body relative to said outer body; a bottom portion of said outer body is configured to releasably engage an outer surface of said tulip; said outer body includes one or more side openings configured to enable a user to a) view movement of one or more portions of said inner body within said outer body, b) view a distance of longitudinal movement of said inner body within said outer body, and/or c) view a positioning of said engagement legs relative to said engagement areas on said upper pressure cap.

17. A polyaxial pedicle screw arrangement that can be temporarily locked in position during a surgical procedure; said polyaxial pedicle screw arrangement comprising a tulip, an upper pressure cap, a locking device, and a lower pressure cap/flexible collet; said upper pressure cap and/or said lower pressure cap/flexible collet are configured to move within said tulip when a force is applied to said upper pressure cap and/or said lower pressure cap/flexible collet by said locking device locking an orthopedic device in said tulip; said upper pressure cap separate component from said lower pressure cap/flexible collet; a lower portion of said upper pressure cap is configured to engage an upper portion of said lower pressure cap/flexible collet, and wherein said movement of said upper pressure cap and/or said lower pressure cap/flexible collet in at least one direction within said tulip is configured to inhibit or prevent movement of a pedicle screw relative to said upper pressure cap and/or said lower pressure cap/flexible collet.

18. The polyaxial pedicle screw arrangement as defined in claim 17, wherein said upper pressure cap and/or said lower pressure cap/flexible collet are configured to move upwardly and downwardly along a longitudinal axis of said tulip, and wherein downward movement of said upper pressure cap and/or said lower pressure cap/flexible collet within said tulip is configured to inhibit or prevent movement of the pedicle screw relative to said upper pressure cap and/or said lower pressure cap/flexible collet.

19. The polyaxial pedicle screw arrangement as defined in claim 17, wherein said upper pressure cap is positioned above said pedicle screw and does not engage said pedicle screw.

20. The polyaxial pedicle screw arrangement as defined in claim 17, wherein said tulip includes body that has a top portion and a bottom portion; said top portion including a top cavity; said bottom portion including a bottom cavity; said top portion including two top side openings configured to receive a portion of an orthopedic rod or other type of medical device; said two top side openings forming first and second upwardly extending arms; said top cavity including a top securing surface configured to receive locking device to entrap the orthopedic rod or other type of medical device in said two top side openings and between said locking device and said upper pressure cap, and while said lower pressure cap/flexible collet is spaced from the orthopedic rod or other type of medical device; said bottom cavity is configured to receive at least a portion of said lower pressure cap/flexible collet and/or said pedicle screw.

21. The polyaxial pedicle screw arrangement as defined in claim 20, wherein said body of said tulip includes a mid-opening that is positioned along a longitudinal axis of said body; said opening forming a passageway between the top and bottom cavities.

22. The polyaxial pedicle screw arrangement as defined in claim 20, wherein said body of said tulip includes one or more side slots that are configured to receive a portion of said upper pressure cap; said one or more side slots are configured to limit upward and downward movement of said upper pressure cap along said longitudinal axis of said tulip when said polyaxial pedicle screw arrangement is fully assembled.

23. The polyaxial pedicle screw arrangement as defined in claim 17, wherein said upper pressure cap includes one or more side slots that are configured to receive a portion of said lower pressure cap/flexible collet; said one or more side slots of said upper pressure cap are configured to limit rotational movement of said lower pressure cap/flexible collet about a central longitudinal axis of said tulip and allow upward and downward movement along said central longitudinal axis of said tulip when said pedicle screw arrangement is fully assembled.

24. The polyaxial pedicle screw arrangement as defined in claim 23, wherein said lower pressure cap/flexible collet includes one or more position flanges that extend upwardly from said body of said lower pressure cap/flexible cap, and wherein said one or more position flanges are configured to be at least partially inserted in one or more side slots of said upper pressure cap when said pedicle screw arrangement is fully assembled.

25. The polyaxial pedicle screw arrangement as defined in claim 17, wherein said body of said lower pressure cap/flexible collet includes one or more slots that extend from a base of said body to a point that is spaced from an upper edge of said body; said one or more slots are configured to enable a lower portion of said body to be compressed together and to reduce a cross-sectional area of said lower portion of said body when a downward force is applied to said lower pressure cap/flexible collet.

26. A method for using a polyaxial pedicle screw arrangement as defined in claim 17; said method comprising:
providing said polyaxial pedicle screw arrangement;
providing a medical instrument for use with a polyaxial pedicle screw arrangement; said medical instrument includes an outer body and an inner body; said inner body is configured to be rotatably positioned in said outer body and be rotatable about a central longitudinal axis of said medical instrument; said inner body is configured to move along a central longitudinal axis of said outer body; a top portion of said inner body extends upwardly from a top portion of said outer body; a top portion of said inner body includes one or more engagement members that are configured to be engaged by a medical tool to facilitate in rotation of said inner body relative to said outer body; a bottom portion of said outer body is configured to releasably engage an outer surface of said tulip; said outer body includes one or more side openings configured to enable a user to a) view movement of one or more portions of said inner body within said outer body, b) view a distance of longitudinal movement of said inner body within said outer body, and/or c) view a positioning of said engagement legs relative to said engagement areas on said upper pressure cap; said medical instrument includes one or more engagement legs that are configured to engage a portion of the polyaxial pedicle screw arrangement;
engaging said polyaxial pedicle screw arrangement with said medical instrument to cause said one or more engagement members to engage said upper pressure cap and/or said lower pressure cap/flexible collet; and
causing said inner body to move downwardly relative to said outer body to cause said upper pressure cap and/or said lower pressure cap/flexible collet to move downwardly in said tulip and transform said polyaxial pedicle screw arrangement from a polyaxial moving arrangement to a monoaxial moving arrangement.

27. A medical instrument for use with a polyaxial pedicle screw arrangement; said medical instrument includes an outer body and an inner body; said inner body is configured to be rotatably positioned in said outer body and be rotatable about a central longitudinal axis of said medical instrument; said inner body is configured to move along a central longitudinal axis of said outer body; a top portion of said inner body extends upwardly from a top portion of said outer body; a top portion of said inner body includes one or more engagement members that are configured to be engaged by a medical tool to facilitate in rotation of said inner body relative to said outer body; a bottom portion of said outer body is configured to releasably engage an outer surface of said tulip; said outer body includes one or more side openings configured to enable a user to a) view movement of one or more portions of said inner body within said outer body, b) view a distance of longitudinal movement of said inner body within said outer body, and/or c) view a positioning of said engagement legs relative to said engagement areas on said upper pressure cap; said medical instrument includes one or more engagement legs that are configured to engage a portion of the polyaxial pedicle screw arrangement.
